(19) United States
(12) Patent Application Publication
(10) Pub. No.: US 2025/0025616 A1
(43) Pub. Date: Jan. 23, 2025

(54) OPHTHALMIC SURGICAL CASSETTES

(71) Applicant: Alcon Inc., Fribourg (CH)

(72) Inventors: Vincent A. BAXTER, Temecula, CA (US); Satish YALAMANCHILI, Irvine, CA (US); David DYK, Foothill Ranch, CA (US); Brian William MCDONELL, Irvine, CA (US); Shawn X. GAO, Irvine, CA (US); Brad Hua CHEN, Mission Viejo, CA (US); Behzad HASHEMI, Irvine, CA (US)

(21) Appl. No.: 18/771,647

(22) Filed: Jul. 12, 2024

Related U.S. Application Data
(60) Provisional application No. 63/594,901, filed on Oct. 31, 2023.
(60) Provisional application No. 63/514,416, filed on Jul. 19, 2023.

(51) Int. Cl. A61M 1/00; A61B 17/00; A61F 9/007

(52) U.S. Cl. CPC A61M 1/72; A61F 9/007; A61M 1/743; A61M 1/774; A61B 2017/00199; A61M 2205/121

(57) ABSTRACT

Embodiments disclosed herein provide a surgical cassette including a housing having a base and a cover coupled to the base, wherein the cover and the base each at least partially define one or more channels. The housing further includes a plurality of ports formed in the base and in fluid communication with at least one of the one or more channels, a plurality of bores formed in the base and configured to receive one of a plurality of valve assemblies, and at least one pump landing formed in the base and configured to receive at least one pump assembly. A plurality of valve assemblies is disposed within the plurality of bores and configured to control fluid flow in the one or more channels of the housing. At least one pump assembly is disposed within the at least one pump landing and configured to provide a source of pressure or vacuum to the one or more channels of the housing.

### CROSS-REFERENCE TO RELATED APPLICATIONS

This application claims benefit of and priority to U.S. Provisional Patent Application No. 63/594,901, filed Oct. 31, 2023, and U.S. Provisional Patent Application No. 63/514,416, filed Jul. 19, 2023. The entire contents of each of these applications are incorporated herein by reference.

### INTRODUCTION

Ophthalmic surgical procedures are often classified as anterior segment surgical procedures, posterior segment procedures, or combined anterior segment and posterior segment procedures (i.e., “combined procedures”). The anterior segment refers to the front-most region of the eye, and includes the cornea, iris, and lens. Thus, anterior segment surgical procedures typically include surgeries performed on the iris and/or lens, such as cataract surgery. The posterior segment refers to the back-most region of the eye that includes the anterior hyaloid membrane and the optical structures behind it, such as the vitreous humor, the retina, the choroid, and the optic nerve. Posterior segment surgical procedures typically include retinal and vitreoretinal surgeries. In certain cases, a patient may have pathologies of the eye requiring both anterior and posterior procedures; in such cases, a combined procedure may be performed.

During anterior and/or posterior segment surgery, tissue fragments and other materials may be aspirated or suctioned out of the eye through, e.g., a hollow needle or cannula. Also, during the procedure, an irrigating or infusion fluid may be pumped into the eye to maintain an intraocular pressure (IOP) and prevent collapse of the eye. A surgical cassette having one or more peristaltic and/or venturi pumps and one or more valve assemblies may be operably coupled with a fluidics module of a surgical console and used to facilitate the aspiration/suction and irrigation/infusion functionalities described above. In general, the one or more valve assemblies of the surgical cassette are operable to control the application of pressure and vacuum generated by the one or more peristaltic pumps during the surgical procedure.

However, conventional surgical cassettes have a number of significant shortcomings including, for example, relatively tight sealing tolerances, relatively high torque required for valve operation, restricted flow capacity, the inability to maintain a secure connection with the surgical console, relatively high fabrication costs and complexity, undesirable fluid displacement during valve operation, and redundancy issues, among others.

Therefore, there is a need for improved surgical cassettes that address at least some of the drawbacks described above.

### BRIEF SUMMARY

The present disclosure relates generally to ophthalmic surgical cassettes, valve assemblies therefor, and methods of use thereof.

In certain embodiments, a surgical cassette is provided which is configured to be coupled to a surgical console for ophthalmic irrigation and/or aspiration during a surgical procedure. The surgical cassette includes a housing having a partition separating a first surface of the housing from a second surface of the housing, a plurality of ports formed in the first surface of the housing, and a plurality of corresponding channels adjoining the second surface of the housing and in fluid communication with the plurality of ports. One or more of the channels is in fluid communication with a source of pressure or vacuum. The surgical cassette includes one or more valve assemblies coupled to the housing and configured to control fluid communication between the plurality of channels of the housing. Each of the one or more valve assemblies includes a valve body having a first end, a second end, and a cylindrical surface connecting the first end and second end. One or more passages are formed in the first end of the valve body. The first end of the valve body seals with the first surface of the housing. The valve body is rotatable about a first axis orthogonal to the first end and relative to the first surface of the housing to align the one or more passages with one or more of the plurality of ports of the housing to open fluid communication between corresponding ones of the plurality of channels. A drive interface is formed on the second end of the valve body and is configured to engage a drive mechanism for rotating the valve body.

In certain embodiments, a surgical cassette is provided which is configured to be coupled to a surgical console for ophthalmic irrigation and/or aspiration during a surgical procedure. The surgical cassette includes a housing having a base, a cover coupled to the base, and a manifold coupled to the cover. A venturi reservoir is disposed inside the housing between the base and cover. The venturi reservoir includes a level sensor area defined in the base for determining a fluid level in the venturi reservoir, a first port disposed through the base on a first side of the level sensor area, a second port disposed through the base on a second side of the level sensor area, and a plurality of air baffles disposed within the venturi reservoir. The plurality of air baffles is configured to divert air bubbles entering the venturi reservoir from each of the first and second ports in the base away from the level sensor area.

In certain embodiments, a surgical cassette is provided which is configured to be coupled to a surgical console for ophthalmic irrigation and/or aspiration during a surgical procedure. The surgical cassette includes one or more valve assemblies coupled to a housing and configured to control fluid communication between a plurality of channels within the housing. Each of the one or more valve assemblies includes a valve body having a first end, a second end, and a cylindrical surface connecting the first end and second end. One or more passages are formed in the first end of the valve body. The valve body is configured to engage with the housing and a retaining ring to form a seal with a first surface of the housing.

In certain embodiments, a system for detecting a surgical cassette when the surgical cassette is coupled to a surgical console is provided. The surgical console includes an image sensor area defined within the surgical console, an image sensor disposed within the image sensor area, and at least one light source disposed within the image sensor area. The surgical cassette includes a cavity defined within the surgical cassette and a plurality of surfaces disposed within the cavity. The at least one light source disposed within the image sensor area is configured to create an optical path through the cavity defined within the surgical cassette.

Embodiments disclosed herein provide a surgical cassette including a housing having a partition separating a first surface of the housing from a second surface of the housing, a plurality of ports formed in the first surface of the housing, and a plurality of corresponding channels adjoining the second surface of the housing and in fluid communication with the ports. One or more of the channels is in fluid communication with a source of pressure or vacuum. The surgical cassette includes one or more valve assemblies coupled to the housing and configured to control fluid communication between the channels. Each valve assembly includes a valve body having a first end, a second end, and a cylindrical surface connecting the first end and second end. One or more passages are formed in the first end of the valve body, which seals with the first surface of the housing.

Embodiments disclosed herein provide a surgical cassette including a housing having a base, a cover coupled to the base, and a manifold coupled to the cover. A venturi reservoir is disposed inside the housing between the base and cover. The venturi reservoir includes a level sensor area defined in the base for determining a fluid level in the venturi reservoir, a first port disposed through the base on a first side of the level sensor area, a second port disposed through the base on a second side of the level sensor area, and a plurality of air baffles disposed within the venturi reservoir. The plurality of air baffles is configured to divert air bubbles entering the venturi reservoir from each of the first and second ports in the base away from the level sensor area.

Embodiments disclosed herein provide a system for detecting a surgical cassette when the surgical cassette is coupled to a surgical console. The surgical console comprises an image sensor area defined within the surgical console, an image sensor disposed within the image sensor area, a window enclosing the image sensor area, and at least one light source disposed within the image sensor area. The surgical cassette comprises a plurality of surfaces disposed within the surgical cassette. At least one light source disposed within the image sensor area is configured to create an optical path through the surgical cassette.

Embodiments disclosed herein provide a surgical cassette for coupling to a surgical console for surgical irrigation or aspiration during a surgical procedure. The surgical cassette comprises a housing, at least one valve assembly rotatably coupled to the housing and configured to control fluid communication between a plurality of channels within the housing, and at least one retaining ring coupled to the housing. The at least one valve assembly comprises a valve body having a first end, a second end, and a cylindrical surface connecting the first end and second end, and one or more passages defined in the first end of the valve body. The valve body is configured to engage with the housing and the at least one retaining ring to form a seal with a first surface of the housing.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1A illustrates an example of an ophthalmic surgical system that may be used to perform ophthalmic procedures on an eye, according to certain embodiments.

FIG. 1B is an example of subsystems of a console of the ophthalmic surgical system of FIG. 1A, according to certain embodiments.

FIG. 2A is a backside isometric view of an example surgical cassette which may be operably coupled to a console of an ophthalmic surgical system, according to certain embodiments.

FIG. 2B is a backside elevation view of the surgical cassette of FIG. 2A, according to certain embodiments.

FIG. 2C is an exploded frontside isometric view of the surgical cassette of FIG. 2A, according to certain embodiments.

FIG. 2D is an exploded backside isometric view of the surgical cassette of FIG. 2A, according to certain embodiments.

FIG. 2E is a frontside elevation view of a base of the surgical cassette of FIG. 2A, according to certain embodiments.

FIGS. 2F-2G are enlarged exploded frontside and backside isometric views, respectively, of a portion of the surgical cassette of FIG. 2A illustrating an example valve assembly having two passages in the valve body, according to certain embodiments.

FIG. 3A is a top side isometric view illustrating an example valve body having two passages, according to certain embodiments.

FIG. 3B is a bottom side isometric view illustrating the valve body having two passages seen in FIG. 3A, according to certain embodiments.

FIG. 3C is a side elevation view illustrating the valve body having two passages seen in FIG. 3A, according to certain embodiments.

FIG. 3D is a top-down perspective view illustrating the valve body having two passages seen in FIG. 3A, according to certain embodiments.

FIG. 3E is a bottom-up perspective view illustrating the valve body having two passages seen in FIG. 3A, according to certain embodiments.

FIG. 4A is a cross-sectional side view illustrating the valve body having two passages seen in FIG. 3A, when seated in a bore of the surgical cassette of FIG. 2A and compressed, according to certain embodiments.

FIG. 4B is a magnified view of FIG. 4A illustrating a cross-sectional view of a first portion of a sealing material disposed on the valve body having two passages, according to certain embodiments.

FIG. 4C is a magnified view of FIG. 4A illustrating a cross-sectional view of a second portion of a sealing material disposed on the valve body having two passages, according to certain embodiments.

FIG. 4D is a magnified view of FIG. 4A illustrating a cross-sectional top portion of the valve body having two passages, according to certain embodiments.

FIGS. 5A-5B are frontside elevation views of a portion of the surgical cassette of FIG. 2A illustrating two different valve positions, according to certain embodiments.

FIG. 6A is a top side isometric view illustrating another example valve body having only one passage, according to certain embodiments.

FIG. 6B is a bottom side isometric view illustrating the valve body having only one passage seen in FIG. 6A, according to certain embodiments.

FIG. 6C is a side elevation view illustrating the valve body having only one passage seen in FIG. 6A, according to certain embodiments.

FIG. 6D is a top-down perspective view illustrating the valve body having only one passage seen in FIG. 6A, according to certain embodiments.

FIG. 7A is a cross-sectional side view illustrating the valve body having only one passage seen in FIG. 6A, when seated in a bore of the surgical cassette of FIG. 2A and compressed, according to certain embodiments.

FIG. 7B is a magnified view of FIG. 7A illustrating a cross-sectional view of a first portion of a sealing material disposed on the valve body having only one passage, according to certain embodiments.

FIG. 7C is a magnified view of FIG. 7A illustrating a cross-sectional view of a second portion of a sealing material disposed on the valve body having only one passage, according to certain embodiments.

FIG. 7D is a magnified view of FIG. 7A illustrating a cross-sectional top portion of the valve body having only one passage, according to certain embodiments.

FIG. 8 is an exploded backside isometric view illustrating an alternative valve assembly, according to certain embodiments.

FIG. 9 is a top-down perspective view of the surgical cassette of FIG. 2A illustrating the backside surfaces of each of a corresponding plurality of bores comprising a surface finish disposed thereon, according to certain embodiments.

FIG. 10 is a magnified cross-sectional view of the interaction between a surgical cassette and a surgical console illustrating an image sensor disposed in the surgical console detecting a barcode disposed on the surgical cassette that is illuminated by a visible light source disposed within the surgical console, according to certain embodiments.

FIG. 11 illustrates a final image obtained by the image sensor seen in FIG. 10 of the barcode disposed on the surgical cassette, according to certain embodiments.

FIG. 12A illustrates an image of the barcode on the surgical cassette obtained by the image sensor used to produce the final image seen in FIG. 10 when a lower left visible light source remains darkened, according to certain embodiments.

FIG. 12B illustrates another image of the barcode on the surgical cassette obtained by the image sensor used to produce the final image seen in FIG. 10 when an upper right visible light source remains darkened, according to certain embodiments.

FIG. 12C illustrates another image of the barcode on the surgical cassette obtained by the image sensor used to produce the final image seen in FIG. 10 when a lower right visible light source remains darkened, according to certain embodiments.

FIG. 12D illustrates another image of the barcode on the surgical cassette obtained by the image sensor used to produce the final image seen in FIG. 10 when an upper left visible light source remains darkened, according to certain embodiments.

FIG. 13 is a magnified cross-sectional view of the interaction between a surgical cassette and a surgical console illustrating an image sensor disposed in the surgical console detecting a fluid level within the surgical cassette by illuminating a reservoir with infrared light, according to certain embodiments.

FIG. 14A is an image sensor reading corresponding to when the reservoir in FIG. 13 is empty, according to certain embodiments.

FIG. 14B is an image sensor reading corresponding to when the reservoir in FIG. 13 is ¼ full, according to certain embodiments.

FIG. 14C is an image sensor reading corresponding to when the reservoir in FIG. 13 is ½ full, according to certain embodiments.

FIG. 14D is an image sensor reading corresponding to when the reservoir in FIG. 13 is ¾ full, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

The present disclosure relates generally to ophthalmic surgical cassettes, valve assemblies therefor, and methods of use thereof.

Conventional ophthalmic surgical cassettes and systems have a number of significant shortcomings, including a lack of compatibility for both anterior and posterior surgical procedures. Currently, there is a performance and feature set discrepancy between anterior and posterior ophthalmic surgical cassettes and systems. Some surgical cassettes offer good posterior performance and feature sets, but are large, overly complicated, and lack the requisite fluidic performance for anterior procedures. For example, the ability of such cassettes to mitigate post occlusion break surge is limited, or even absent. Meanwhile, other cassettes offer excellent anterior procedure fluidic performance (including fast response to, and mitigation of, post occlusion break surge), but do not provide larger feature sets for posterior surgical procedures. For example, such cassettes do not support fluid air exchange operations, which require high suction flow rates as provided by, e.g., venturi pumps, nor do they support simultaneous use of both a vitrectomy probe and a phacoemulsification handpiece. There also remains room for fluidics improvement of current anterior surgical systems, such as to reduce surge disturbances in the eye by further increasing response and flow capabilities.

Other performance limitations for both anterior and posterior surgical systems include irrigation/infusion flow accuracy, pressure/vacuum sensor sensitivity to mechanical disturbances, interference fit valves with high operational torque, valves with limited fluid flow capacity, complex fluidic schematics with large fluid volumes in fluidic communication to the eye during venting operations, and large cassette layouts to support the complexity of reservoirs, sensors, valves, pumps, and accessory connections, among others.

The present disclosure provides improved surgical cassettes, which address the drawbacks described above.

Certain embodiments disclosed herein provide a surgical cassette that functions as an interface between a patient and a surgical console used to perform a variety of different ophthalmic surgeries. For example, the surgical cassette, in conjunction with the surgical console and its associated console fluidics module, can be utilized for maintaining surgical space within the patient's eye via fluidics balancing during anterior and posterior ophthalmic surgical procedures.

Certain embodiments disclosed herein provide greater flexibility in the layout of the surgical cassette including, e.g., the arrangement of valve assemblies and flow channels therein. For example, when compared to conventional surgical cassettes, certain embodiments disclosed herein provide greater flexibility in valve positioning/placement within the cassette, additional ports connecting to each valve, ports having greater flow area, ports having a variety of shapes, multiple flow connections for a single valve, reduction in size of the surgical cassette, and readily adaptable to injection molding.

Certain embodiments disclosed herein provide valve assemblies for a surgical cassette with improved sealing. For example, embodiments herein disclose valve assemblies having a scaling interface between planar surfaces. In certain embodiments, a valve body has a planar end surface perpendicular to a cylindrical outer surface of the valve body that seals with a corresponding surface of the cassette housing. Because the sealing interface is on a longitudinal end of the valve body, this arrangement may be referred to herein as “end-sealing.” At least in part due to the sealing interface being on the end of the valve body, and the sealing surface engaging with a plastic or plastic composite material of the surgical cassette base, embodiments disclosed herein provide valve assemblies requiring lower sealing tolerances and lower torque for valve operation when compared to conventional valves with a cylindrical sealing interface.

Certain embodiments disclosed herein provide lower fabrication cost and complexity when compared to conventional valves. For example, certain embodiments disclosed herein eliminate the need for slides used in the injection molds for conventional valve and cassette bodies, eliminate the need to plug holes in the cassette body created by the slides, and provide flow channels in any direction without regard to slide capabilities and considerations. In addition, certain embodiments disclosed herein are more easily molded and assembled (e.g., ultrasonically welded, bonded, or snap fit) when compared to conventional valves.

Certain embodiments disclosed herein provide improved valve operation including increased flow capacity and ability to relieve pressure or vacuum to one port without inducing a significant pressure or vacuum disturbance to another port, which is beneficial for post-occlusion break surge mitigation. In certain embodiments, dual path venting is enabled to provide venting to two separate channels simultaneously.

In particular embodiments, a rotatable aspiration valve of a surgical cassette is provided, the rotatable aspiration valve including a plurality of ports, the plurality of ports including a first port, a second port, and a third port. The first port is in fluidic communication with an aspirating handpiece, the second port is in fluidic communication with an aspirating pump disposed within a base of the surgical cassette, and the third port is in fluidic communication with a fluid chamber formed between the base and a cover of the surgical cassette. The aspiration valve is configured to form at least one valve pathway in the surgical cassette. The aspiration valve is further switchable from a first state to a second state. In the first state, the aspiration valve fluidically connects the aspirating handpiece to the aspirating pump. In the second state, the aspiration valve fluidically connects the aspirating handpiece to the fluid chamber for venting a fluidic volume in fluidic communication with the aspirating handpiece. In certain embodiments, the aspiration valve is configured to have a first valve pathway and a second valve pathway. In such embodiments, in the second state, the aspiration valve connects the first valve pathway, which connects from the aspirating handpiece to the fluid reservoir, with the second valve pathway, which connects the aspirating pump to the fluid reservoir, for separate venting of the fluidic volume in fluidic communication with the aspiration pump.

In particular embodiments, a surgical cassette for ophthalmic irrigation/infusion and/or aspiration/suction during a surgical procedure is provided. The surgical cassette includes a base. The base is configured to adjoin to a first cover to facilitate anterior surgical performance, or to adjoin to a second cover to facilitate both anterior and posterior surgical performance.

Generally, the surgical cassettes described herein provide an improvement of fluidics performance (including surge mitigation) for both anterior and posterior procedures in a size reduced form factor, which is facilitated by, for example: a low, exposed vent volume fluidic arrangement; one or more vent valve ports positioned directly over a fluidic chamber; dual vent and low friction valves; alignment features disposed around pressure sensor diaphragms that engage pressure sensors for stable pressure readings; pressure sensors positioned at the center of pumps, adjacent irrigation and aspiration pumps; four face-sealed valve assemblies positioned in corner placements of the cassettes; a fluidic chamber with fluid-directing baffling; a total internal reflection fluid level sensor with barcode reading capabilities; a venturi port providing air connection from the fluidic chamber to a venturi pump in a surgical console; an air filter with drain mitigation between a venturi port and fluid chamber; and, a redundant irrigation pressure sensor in fluidic communication with an irrigation pump and first irrigation pressure sensor. These features, as well as others, are described below in more detail.

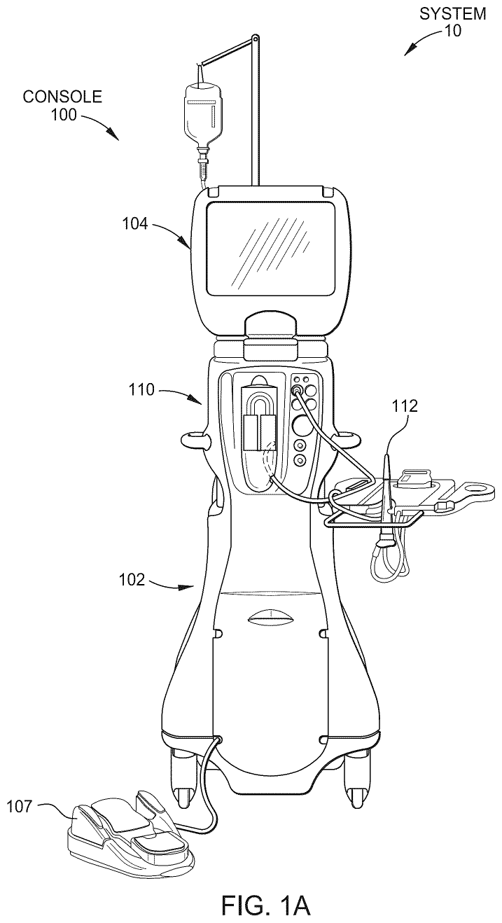

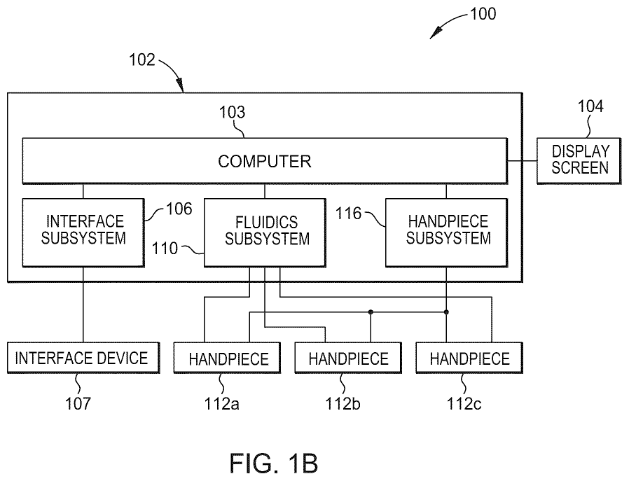

FIG. 1A illustrates an example of an ophthalmic surgical system 10 that may be used to perform ophthalmic procedures on an eye, according to certain embodiments. In the illustrated embodiments, system 10 includes console 100 (also referred to as a “surgical console”), a housing 102, a display screen 104, an interface device 107 (e.g., a foot pedal), a fluidics subsystem 110, and a handpiece 112, coupled as shown and described in more detail with reference to FIG. 1B.

FIG. 1B is an example of subsystems of console 100 of ophthalmic surgical system 10 of FIG. 1A, according to certain embodiments. Console 100 includes housing 102, which accommodates a computer 103 (with an associated display screen 104) and subsystems 106, 110, and 116, which support interface device 107 and handpieces 112 (112a-c). An interface device 107 receives input to surgical system 10, sends output from system 10, and/or processes the input and/or output. Examples of an interface device 107 include a foot pedal, manual input device (e.g., a keyboard), and a display. Interface subsystem 106 receives input from and/or sends output to interface device 107.

Handpiece 112 may be any suitable ophthalmic surgical instrument, e.g., an ultrasonically-driven phacoemulsification (phaco) handpiece, a laser handpiece, an irrigating cannula, a vitrectomy handpiece, or another suitable surgical handpiece. Fluidics subsystem 110 provides fluid control for one or more handpieces 112 (112a-c). For example, fluidics subsystem 110 may manage fluid for an irrigating cannula. Handpiece subsystem 116 supports one or more handpieces 112. For example, handpiece subsystem 116 may manage ultrasonic oscillation for a phaco handpiece, provide laser energy to a laser handpiece, control operation of an irrigating cannula, and/or manage features of a vitrectomy handpiece.

Computer 103 controls operation of ophthalmic surgical system 10. Generally, computer includes a processor and a memory. The memory may include any device operable to receive, store, or recall data, including, but not limited to, electronic, magnetic, or optical memory, whether volatile or non-volatile. The memory may include code stored thereon. The code may include instructions that may be executable by the processor. The code may be created, for example, using any programming language, including but not limited to, C++ or any other programming language (including assembly languages, hardware description languages, and database programming languages). In some instances, the code may be a program that, when loaded into the processor, causes the surgical console to receive and process information from one or more of subsystems 106, 110, and 116, for, e.g., providing fluid control for one or more handpieces 112.

The processor may be, or include, a microprocessor, a microcontroller, an embedded microcontroller, a programmable digital signal processor, or any other programmable device operable to receive information from the memory or other devices in communication with the processor, computer 103, and/or console 100, and perform one or more operations on the received information. For example, the processor may send instructions to components of fluidics subsystem 110, or other devices or systems in communication with computer 103, for controlling such devices and systems. The processor may also be operable to output results based on the operations performed thereby. A display screen 104 shows data and other output results provided by the processor of computer 103. In some instances, the processor may also be or include an application specific integrated circuit, a programmable gate array, programmable array logic, or any other device of combinations of devices operable to process electric signals.

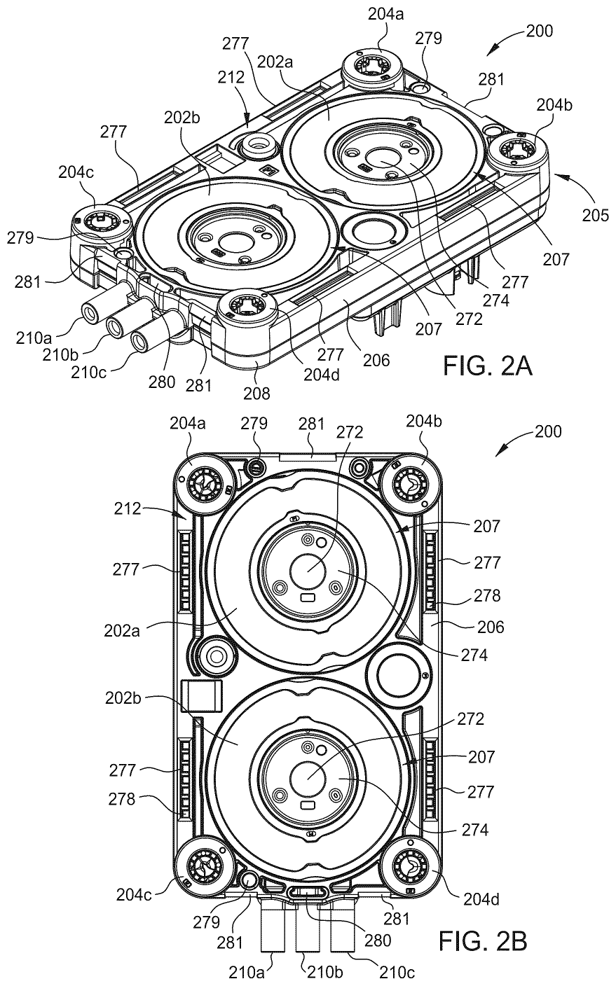

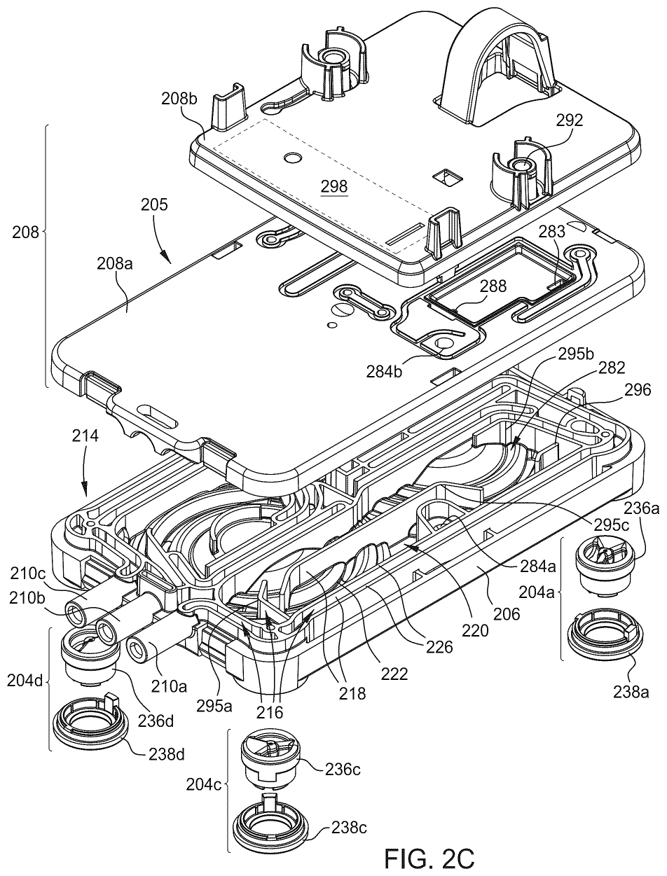

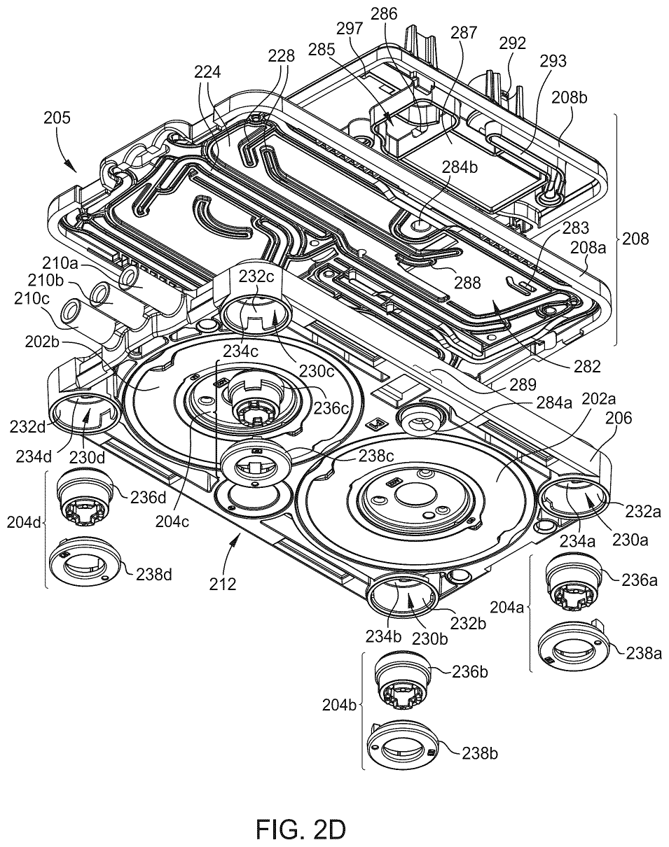

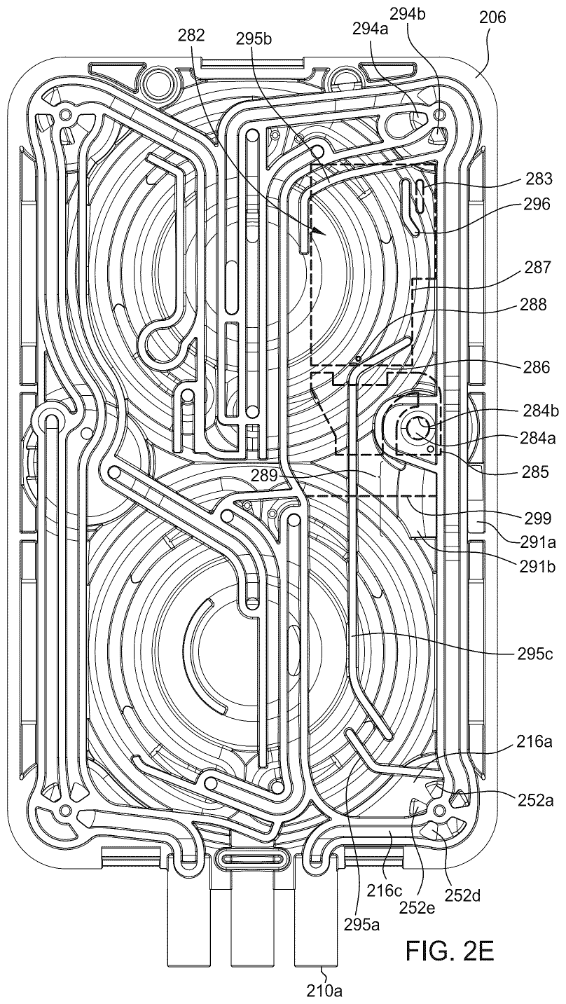

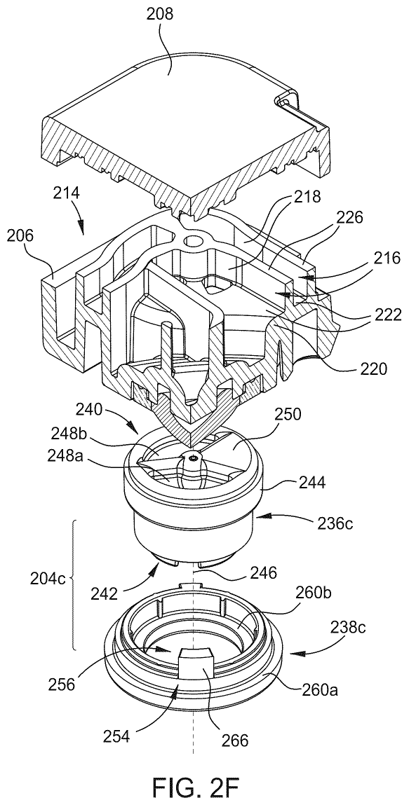

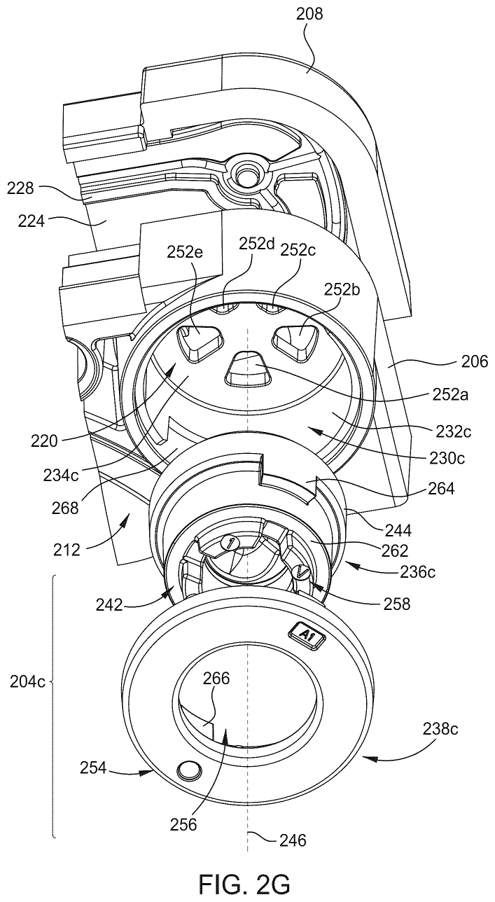

FIG. 2A is a backside isometric view of an example surgical cassette 200 which may be operably coupled to a console of an ophthalmic surgical system (e.g., console 100 of ophthalmic surgical system 10 illustrated in FIGS. 1A-1B), according to certain embodiments. FIG. 2B is a backside elevation view of surgical cassette 200 of FIG. 2A, according to certain embodiments. FIGS. 2A-2B are described together herein for clarity. Surgical cassette 200 includes two adjacent pump assemblies 202 (202a-b,), which provide a source of pressure and/or vacuum within the surgical cassette 200, and four valve assemblies 204 (204a-d), each adjacent to a pump assembly and adjacent to two cassette edges, where the four valve assemblies control pressure and/or fluid communication within surgical cassette 200. In certain embodiments and as further described below, surgical cassette 200 may also include two pressure sensor diaphragms 272 and two diaphragm retaining rings 274, each located at the center of a pump assembly 202, where the pressure sensor diaphragms 272 are used to allow measurement of pressure and/or vacuum within the surgical cassette 200. In certain embodiments, the diaphragm retaining rings 274 incorporate alignment features for engagement to pressure sensors on the console 100. The alignment features may be conical and recessed or may be a group of features including a conical recess, a v-groove recess, and a flat surface positioned around the center of the diaphragm 272. In certain embodiments, an elastomer diaphragm for redundant pressure measurement is adjacent to the two pump assemblies. In certain other embodiments, there may be only one pump assembly 202 or more than two pump assemblies 202. In certain other embodiments, there may be more or less than four valve assemblies 204 (e.g., two to six valve assemblies).

Surgical cassette 200 has a housing 205 including a base 206, a cover assembly 208 coupled to base 206, and inlet/outlet ports 210 (210a-c) in base 206 which provide pressure and/or fluid communication between inside and outside of the housing 205. Fluid lines (e.g., tubing) may be coupled between each port 210a-c and a corresponding component of fluidics subsystem 110 and/or a corresponding handpiece 112a-c (shown in FIGS. 1A-1B) or other device. In certain embodiments, the base 206 and cover 208 form a rectangular shape with rounded corners, allowing the base 206 and cover 208a to circumscribe up to four valves at each of the rounded corners.

In certain embodiments, one of a first pump assembly 202a or second pump assembly 202b provides a source of pressure (e.g., to create a driving force for fluid irrigation) while the other one of the first pump assembly 202a or second pump assembly 202b provides a source of vacuum (e.g., to create suction for fluid aspiration) within the surgical cassette 200. The first pump assembly 202a and second pump assembly 202b may thus be peristaltic pumps or any other suitable type of pump for generating pressure and/or vacuum. In certain embodiments, each of the first pump assembly 202a and second pump assembly 202b are in fluid communication with one of the inlet/outlet ports 210 to facilitate pressure and/or fluid communication between inside and outside of the housing 205. For example, at least one of the first pump assembly 202a and second pump assembly 202b may be in fluid communication with the port 210a for aspiration/suction, and/or at least one of the first pump assembly 202a and second pump assembly 202b may be in fluid communication with the port 210c for irrigation/infusion.

In certain embodiments, an external source of pressure and/or vacuum is coupled to surgical cassette 200. In such embodiments, the external source may be either in place of or in addition to pump assemblies 202. In certain embodiments, the external source makes a fluidic connection from the console 100 to the surgical cassette 200 through a venturi port with an elastomer seal, described in more detail below, on the face of the surgical cassette 200. In certain embodiments, each of the first pump assembly 202a and the second pump assembly 202b is configured to engage with one or more rollers of a roller pump disposed on a surgical console, such as console 100. For example, when the surgical cassette 200 is coupled to the console 100, an elastomeric surface of each pump assembly 202a, 202b may be contacted against one or more rollers of a roller pump on the console 100. Thereafter, during use, manual forces provided by the rollers rolling along and pressing against the elastomeric surface may drive fluid within the corresponding pump assembly 202a or 202b, and thus within surgical cassette 200, to generate pressure or vacuum as needed.

In certain embodiments, the first pump assembly 202a and second pump 202b assembly are identical to each other. In certain embodiments, the first pump assembly 202a and second pump 202b assembly are disposed adjacent to each other, either centrally or peripherally along the base 206. In certain embodiments, each of the pump assemblies 202 is disposed within a pump landing 207, or recess, formed in the base 206. The pump landing 207 may include one or more features for coupling the pump assembly 202 to the base 206, in addition to one or more fluid channels configured to facilitate the provision of pressure or vacuum to the surgical cassette 200.

Valve assemblies 204 are coupled to base 206. Valve assemblies 204 function cooperatively to control pressure and/or fluid communication within and through surgical cassette 200. In the illustrated embodiments, surgical cassette 200 includes a first valve assembly 204a, a second valve assembly 204b, a third valve assembly 204c, and a fourth valve assembly 204d.

In certain embodiments, each of the valve assemblies 204 may be centrally disposed on the base 206, e.g., adjacent to the pump assemblies 202. In certain embodiments, each of the valve assemblies 204 may be peripherally arranged along the base 206, such as in one or more corners of the base 206. For example, as shown in the embodiments of FIG. 2A, the four valve assemblies 204 are arranged at the four corners of housing 205 and surrounding the two pump assemblies 202, which are arranged towards a center of housing 205. However, in other embodiments, pump assemblies 202 and valve assemblies 204 may have any other suitable arrangement.

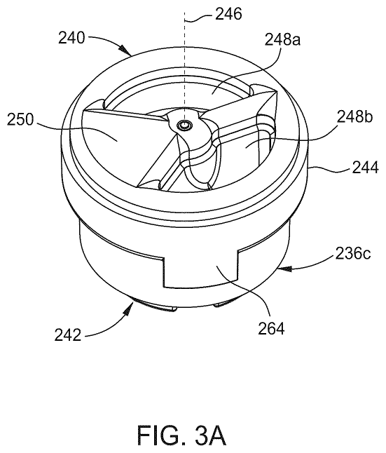

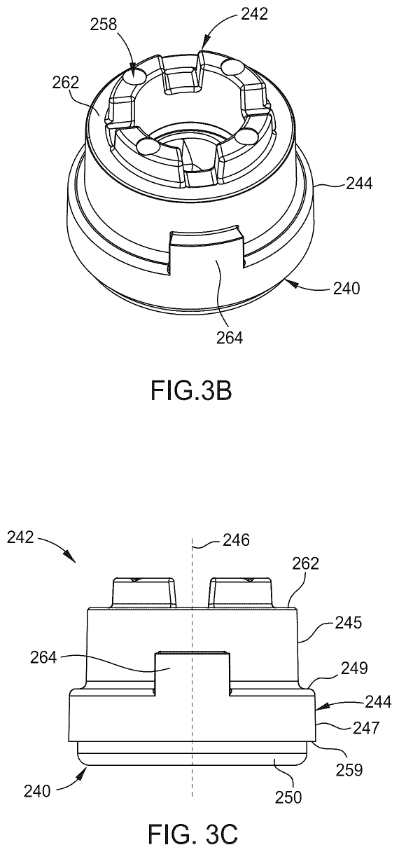

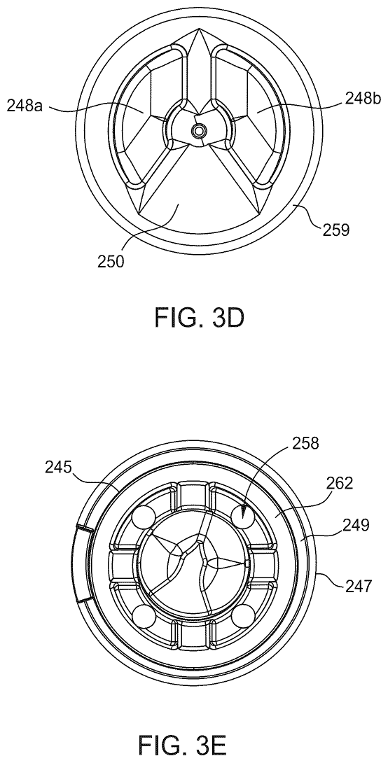

In certain embodiments, valve assemblies 204 may be operated to route fluid flow selectively between multiple channels of housing 205 as described in more detail below with respect to FIGS. 3A-3B. For example, first valve assembly 204a and third valve assembly 204c may be in pressure and/or fluid communication with first pump assembly 202a and port 210a to provide aspiration/suction through port 210a during an operation, and second valve assembly 204b and fourth valve assembly 204d may be in pressure and/or fluid communication with second pump assembly 202b and port 210c to provide irrigation/infusion through port 210c during the same operation. In certain embodiments, port 210b is an administration port, and fluid that is to be sent to the patient's eye via the port 210c may be first flowed into the cassette 200 via the admin port 210b. In certain embodiments, port 210b may be an auxiliary port that is inactive during the operation and instead may be used to provide maintenance access for servicing surgical cassette 200 between operations.

The surgical cassette 200 further incorporates clamping and alignment features for mechanical coupling to the console. In one embodiment, two pairs of vertical clamping slots 277 are disposed symmetrically about the adjacent pump assemblies 202 in the base 206. Clamping pads 278 may be disposed internally in the base 206 at each end of the clamping slots 277 and provide contact points for a clamping mechanism within the console 100. In certain embodiments, a hole 279 and/or a slot 280 are disposed near the lower and/or upper edges of the base 206, which engage and align with pins in the console 100 when the surgical cassette 200 is clamped thereto. Retaining features 281 are disposed at the top and bottom edges of the base 206 and may interface with spring loaded retaining arms in the console to keep the surgical cassette secured after the clamp is disengaged.

Pump assemblies 202 and valve assemblies 204 are located on a backside 212 of base 206, which is visible in FIGS. 2A-2B. Cover assembly 208 is coupled to a frontside 214 (shown in FIG. 2C) of base 206 which faces away from backside 212. In certain embodiments, cover assembly 208 may be welded, bonded, or fastened to base 206 using any suitable coupling mechanism. For example, cover assembly 208 may be coupled to base 206 using a solid-state welding technique (e.g., ultrasonic welding in which high-frequency mechanical vibrations are locally applied to parts being held together under pressure to create a solid-state weld). Backside 212 of base 206 is configured to interface with console 100 when surgical cassette 200 is coupled thereto. For example, a drive interface on a valve body of each valve assembly 204 may engage a corresponding drive mechanism of fluidics subsystem 110 within console 100 for rotating the corresponding valve body. The valve body is described in more detail below with respect to FIGS. 2F-2G. In certain embodiments, the drive mechanism is a direct drive mechanism, wherein the drive interface engages with a motor (e.g., without gears) that operates at lower torque with faster valve response time when compared to a geared drive motor that is conventionally used. In such embodiments, the valve body may be rotated in a one-to-one ratio with the drive motor. However, the embodiments described herein may use any suitable type of drive motor. The components of surgical cassette 200 are described in more detail below with respect to FIGS. 2C-2D.

FIG. 2C is an exploded frontside isometric view of surgical cassette 200 of FIG. 2A, according to certain embodiments. FIG. 2D is an exploded backside isometric view of surgical cassette 200 of FIG. 2A, according to certain embodiments. FIGS. 2C-2D are described together herein for clarity. In the illustrated embodiments, cover assembly 208 includes two separate pieces including first cover piece 208a (also referred to as a “cover”) coupled directly to frontside 214 of base 206 and second cover piece 208b (also referred to as a “manifold,” which may serve, in part, to identify a configuration or type of the surgical cassette 200 and/or provide a handle for a user) coupled to cover 208a. In some other embodiments, cover assembly 208 may consist of only a single piece. Generally, each of the base 206 and cover assembly 208 (as either a single piece or two separate pieces) comprise substantially planar and parallel components when assembled together.

A plurality of channels 216 are formed in housing 205. The plurality of channels 216 are arranged to provide a plurality of independent fluid paths between pump assemblies 202, valve assemblies 204, and inlet/outlet ports 210. Each channel 216 is longitudinally and equatorially defined in a first and second direction generally parallel to a plane of housing 205. Each channel 216 includes sidewalls 218 oriented perpendicular to the plane of housing 205 and enclose the corresponding channel 216 therebetween. In addition, a depth of each channel 216 is defined in this perpendicular direction between a partition 220 (also referred to as a “lower wall”) of base 206 (e.g., frontside surface 222 thereof) and an inner surface 224 (shown in FIG. 2D) of cover 208a. The plurality of channels 216 are sealingly enclosed through contact between a first surface 226 formed on frontside 214 of base 206 and a corresponding face 228 (shown in FIG. 2D) extending from inner surface 224 of cover 208a. In order to effect sealing, first surface 226 and corresponding face 228 of cover 208a are molded to include matching contours and assembled to ensure precise alignment between opposing sides. In some other embodiments, the plurality of channels 216 may be formed in cover 208a instead of base 206 and sealingly enclosed through contact between a backside surface of cover 208a and a corresponding frontside surface 222 of base 206. In such embodiments, base 206 and cover 208a may be fabricated by additive manufacturing methods.

Four bores 230 (230a-d) are formed in backside 212 of base 206 for receiving a corresponding valve assembly 204. Each bore 230 is defined by a cylindrical inner wall 232 (232a-d) and a backside surface 234 (234a-d) of partition 220. In certain other embodiments, there may be more or less than four bores to correspond to each valve assembly. One or more ports (e.g., five shown in FIGS. 2F-2G) are formed through partition 220. The ports connect frontside surface 222 and opposite backside surface 234 of partition 220. Each port corresponds to one of the plurality of channels 216 in contact with, or adjoining, frontside surface 222 of partition 220. The ports are described in more detail below with respect to FIGS. 2F-2G and 3A-3B.

Each valve assembly 204 generally includes a valve body 236 (236a-d) and a retaining ring 238 (238a-d) that is configured to be disposed in a corresponding bore 230. The valve body 236 and retaining ring 238 fit together in a stacked arrangement. The valve body 236 is disposed between backside surface 234 and the corresponding retaining ring 238. The retaining ring 238 applies a retention force on the corresponding valve body 236 to press the valve body 236 against backside surface 234 as described in more detail below. In some other embodiments, instead of being defined within base 206, each bore 230 may be defined within a corresponding retaining ring 238 that fits around a corresponding valve body 236. In some other embodiments, each valve body 236 may be rotatably coupled to base 206 using a retention cap. The retention cap may be fixedly disposed through valve body 236 (e.g., aligned with a longitudinal axis of valve body 236). In such embodiments, each valve body 236 may be coupled to base 206 without being disposed in a corresponding bore 230.

A venturi reservoir 282 is disposed inside housing 205 of surgical cassette 200 between base 206 and cover 208a. Venturi reservoir 282 serves at least two primary functions that are integral to the operation of surgical cassette 200. In general, venturi reservoir 282 provides a connection to a vacuum source for suction of fluids during a venturi operation, provides a fluid volume sink for normal vacuum venting within the surgical cassette, and provides a fluid volume sink for venting vacuum pressure that may build-up within surgical cassette 200 in the event of a post-occlusion break surge. In certain embodiments, the venturi reservoir 282 also allows air to separate from liquid and then evacuate out the cassette 200 through the surgical console during normal aspiration/suction use with venturi vacuum.

FIG. 2E is a frontside elevation view of base 206, according to certain embodiments. Note that in FIG. 2E, some parts of cover 208a and manifold 208b are shown in phantom for illustrative purposes. Although some aspects of venturi reservoir 282 are visible in FIGS. 2C-2D, certain aspects are illustrated more clearly in FIG. 2E. FIGS. 2C-2E are, therefore, described together herein for clarity.

Surgical cassette 200 may be coupled to an external vacuum source (e.g., a venturi source) disposed in console 100. Vacuum pressure from the external vacuum source is applied to venturi reservoir 282 through a vacuum port 283 in cover 208a. In the illustrated embodiments, vacuum port 283 is an elongated slot. In certain embodiments, vacuum pressure within venturi reservoir 282 is about 720 mmHg (millimeters of mercury). In certain embodiments, maximum airflow through vacuum port 283 is about 1.2 standard liters per minute. The external vacuum source is configured to apply vacuum pressure to venturi reservoir 282 through a vacuum flow path that passes from upstream to downstream through vacuum port 283 in cover 208a and into manifold 208b where the air is filtered before passing back through respective openings in cover 208a and base 206 before reaching the external vacuum source. The vacuum flow path is described in more detail below.

In certain embodiments, an opening 284a in backside 212 of base 206 is coupled to a vacuum port on console 100 leading to the external vacuum source. Opening 284b in cover 208a is aligned with opening 284a in base 206. Openings 284a-b are oriented in a direction perpendicular to a plane of base 206. Openings 284a-b are in pressure communication between the external vacuum source and a catch reservoir 285 that is defined between cover 208a and manifold 208b. Catch reservoir 285 is in fluid communication, through opening 286, with a filter 287 that is disposed between cover 208a and manifold 208b. Catch reservoir 285 collects liquid that leaks through filter 287 and prevents the unexpectedly leaked liquid from entering the vacuum source.

Filter 287 is in pressure communication between the external vacuum source and vacuum port 283 in cover 208a. The location of filter 287 overlays at least a portion of venturi reservoir 282 enabling the use of a more compact housing 205 compared to other designs. An upstream side of filter 287 faces towards cover 208a. A downstream side of filter 287 faces towards manifold 208b. Filter 287 seals with manifold 208b to prevent liquid from leaking around filter 287. Filter 287 permits air to pass from vacuum port 283 to catch reservoir 285 in manifold 208b while blocking liquid from passing to the downstream side of filter 287 and into the external vacuum source. In certain embodiments, filter 287 is directly coupled to manifold 208b (e.g., ultrasonically welded). In certain embodiments, filter 287 is hydrophobic, and thus impermeable to aqueous fluids.

In certain cases, liquid is able to pass through vacuum port 283 and becomes trapped between cover 208a and the upstream side of filter 287. A filter drain hole 288 is disposed through cover 208a in a position overlaying a lower end of filter 287. Filter drain hole 288 is in fluid communication between the upstream side of filter 287 and venturi reservoir 282 to drain the trapped liquid back into venturi reservoir 282. Draining of the trapped liquid through filter drain hole 288 helps maintain maximum usable filter area for maximum air flow through vacuum port 283. In certain embodiments, filter drain hole 288 is much smaller than vacuum port 283 in terms of cross-sectional area to prevent or reduce fluid contained in venturi reservoir 282 from easily passing through filter drain hole 288 in the direction of filter 287. In certain embodiments, the cross-sectional area of filter drain hole 288 is about 10% or less of the cross-sectional area of vacuum port 283.

Venturi reservoir 282 includes a level sensor area 289 defined in base 206. Vacuum port 283 is disposed above level sensor area 289. Level sensor area 289 is disposed along an optical path of a light sensor in console 100 that is used to determine a fluid level in venturi reservoir 282. In certain embodiments, the light sensor is an infrared sensor, a single camera sensor, or a complementary metal oxide semiconductor (CMOS) sensor. During normal operation, a nominal fluid level (indicated by dashed line 299) in venturi reservoir 282 is between lower and upper limits of level sensor area 289. The cavity corresponds to level sensor area 289. The relatively small size or footprint of level sensor area 289 enables the use of a more compact housing 205 compared to other designs.

In certain embodiments, venturi reservoir 282 receives fluids that are aspirated through port 210a of base 206. For example, aspirated fluids may enter port 210a, flow through channel 216c to port 252d, and then enter into venturi reservoir 282 through port 252e. Thus, port 252e is in fluid communication with an aspiration line of, e.g., surgical handpiece 112 (shown in FIG. 1A) or other device through port 210a of base 206. Port 252e is disposed through base 206 below level sensor area 289. Note that ports 252d-e are also referred to as “fourth port” and “fifth port,” respectively, with regard to the description of the valve in conjunction with FIGS. 3A-3B.

In certain embodiments, venturi reservoir 282 receives fluids that are suctioned through port 292 on the frontside of manifold 208b. For example, fluids may enter port 292, flow through channel 293 in manifold 208b to port 294a in base 206, and then enter into venturi reservoir 282 through port 294b. Port 294b is disposed through base 206 above level sensor area 289. In certain embodiments, fluids entering venturi reservoir 282 through port 252e or port 294b include a mixture of liquid, such as BSS (Balanced Salt Solution), and air. In some other embodiments, the fluid is only liquid or only air. In certain embodiments, a flow rate of the fluid is about 200 cc/min (cubic centimeter per minute) or less. The presence of air bubbles in or near level sensor area 289 interferes with accurate detection of the fluid level in venturi reservoir 282 because the air bubbles obscure the air-liquid interface. Therefore, in certain embodiments, a plurality of air baffles 295 (295a-c) are disposed within venturi reservoir 282 in order to divert air bubbles away from level sensor area 289. The plurality of air baffles 295 are integral with base 206 and contact cover 208a when cover 208a is coupled to base 206. In some other embodiments, the plurality of air baffles 295 are integral with cover 208a instead of base 206.

A lower air baffle 295a is disposed above port 252e and extends up and to the left of the viewer in FIG. 2E. An upper air baffle 295b is disposed below port 294b and extends down and to the left of the viewer in FIG. 2E. Air baffles 295a-b form corresponding channel-like structures within venturi reservoir 282 that begin at corresponding ports 252e, 294b and end on the left side of venturi reservoir 282. For example, first channel 216a corresponds to a portion of venturi reservoir 282 below lower air baffle 295a. A central air baffle 295c is located to the right of the respective ends of air baffles 295a-b and extends from below to above level sensor area 289. The position of central air baffle 295c prevents air bubbles from crossing level sensor area 289 even after the air bubbles pass above lower air baffle 295a and below upper air baffle 295b, respectively. An opening between lower air baffle 295a and central air baffle 295c provides a path for equalization of fluid on the left and right sides of central air baffle 295c, so that the fluid level in level sensor area 289 corresponds to the actual fluid level in venturi reservoir 282. An opening between upper air baffle 295b and central air baffle 295c provides a path for the flow of air bubbles from the left side of central air baffle 295c to vacuum port 283 without crossing level sensor area 289.

A liquid baffle 296 is disposed within venturi reservoir 282 above level sensor area 289. Liquid baffle 296 is configured to reduce or prevent liquid from entering vacuum port 283 in cover 208a, such as during bubbling, frothing, or overrun of the liquid contained in venturi reservoir 282. Like air baffles 295, liquid baffle 296 is integral with base 206 and contacts cover 208a when cover 208a is coupled to base 206. An upper portion of central air baffle 295c extends directly below liquid baffle 296 so that liquid falling down from liquid air baffle 296 is blocked from entering the right side of central air baffle 295c and subsequently prevented from crossing level sensor area 289.

FIGS. 2F-2G are enlarged exploded frontside and backside isometric views, respectively, of a portion of surgical cassette 200 of FIG. 2A illustrating an example valve assembly having two recessed passages in the valve body, according to certain embodiments. FIGS. 2F-2G and 3A-3E are described together herein for clarity.

Valve body 236c of third valve assembly 204c has a first end 240, a second end 242, a cylindrical outer surface 244 connecting first end 240 and second end 242, and a longitudinal axis 246 orthogonal to first end 240. Cylindrical outer surface 244 includes multiple stepped portions having different outer dimensions. In some embodiments, the cylindrical outer surface 244 is comprised of a base portion 245 separated from a collar portion 247 by a first shoulder 249, the collar portion 247 having a larger diameter relative to the base portion 245 as best seen in FIG. 3C. In one embodiment, the base portion 245 has a diameter of approximately 13 mm (millimeters) and a longitudinal length of 5.21 mm, while the collar portion has a diameter of approximately 16 mm and a longitudinal length of 3.38 mm. Transitioning from the collar portion 247 to a sealing material 250 is a second shoulder 259.

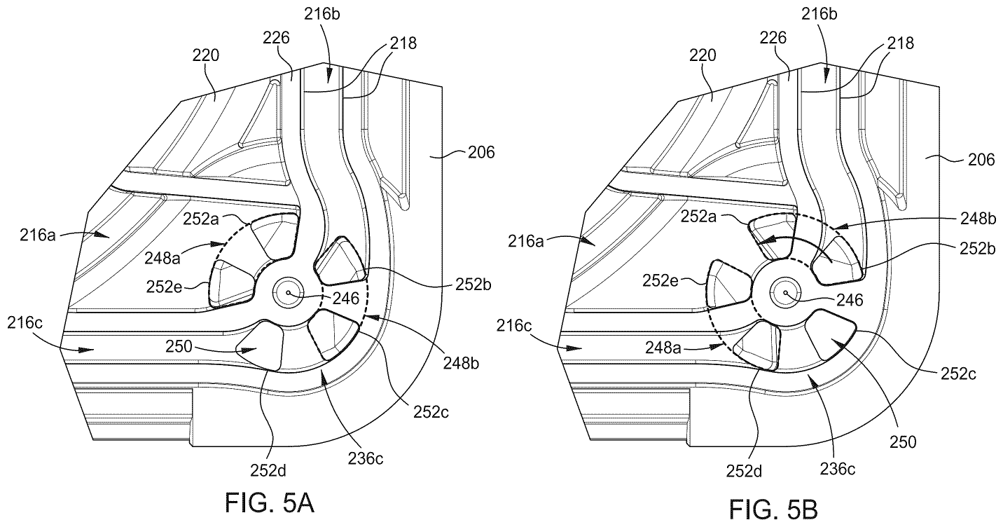

Valve body 236c is rotatable about longitudinal axis 246. Two passages 248 (248a-b) are formed in valve body 236c at first end 240. In FIG. 2F, first passage 248a and second passage 248b are about equal in length when measured in a circumferential direction about longitudinal axis 246 (e.g., extending about longitudinal axis 246 in a circumferential direction by about 140°-150°. In the illustrated embodiments, each passage 248 is sized to simultaneously open fluid communication with two ports of base 206 as described in more detail below respect to FIGS. 5A-5B. In some other embodiments, each passage 248 may be sized to simultaneously open fluid communication with any suitable number of ports (e.g., two, three, or four ports). In the illustrated embodiments, passages 248 include arc-shaped annular segments extending circumferentially about longitudinal axis 246. In certain embodiments, a cross-section of passages 248 may be circular, round, oval, polygonal, square, any other suitable shape, or combinations thereof. Terminal ends of each passage 248 are defined at first end 240 of valve body 236c. In certain embodiments, a center axis of each passage 248 at the terminal ends is parallel to longitudinal axis 246. In certain embodiments, at least a portion of each passage 248, e.g., the portion between the terminal ends, is orthogonal to longitudinal axis 246. In certain embodiments, during fabrication, passages 248 are machined or molded in a direction parallel to longitudinal axis 246, e.g., starting from first end 240. In other words, an entire surface of each passage 248 is visible from first end 240 when viewed in a direction parallel to longitudinal axis 246. In the illustrated embodiments, passages 248 include an equal flow area. In some other embodiments, passages 248 may have different flow areas. In certain other embodiments, there may be only one passage or more than two passages formed in the valve body.

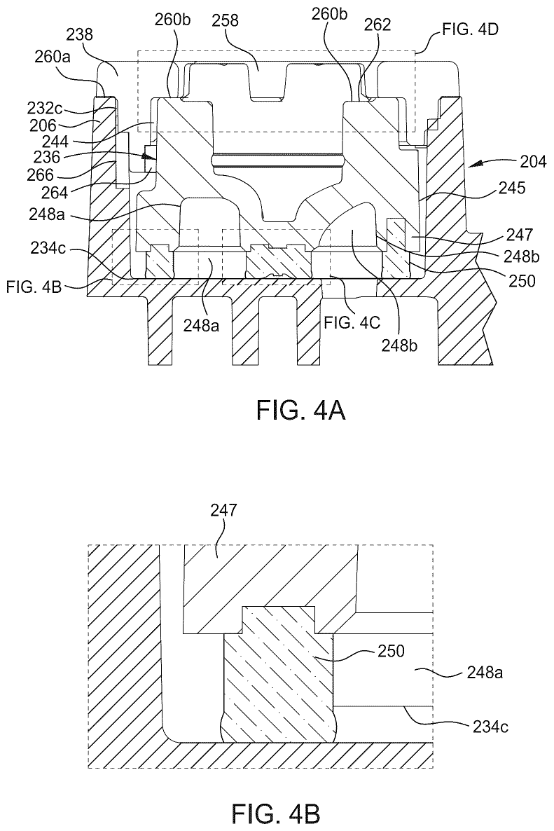

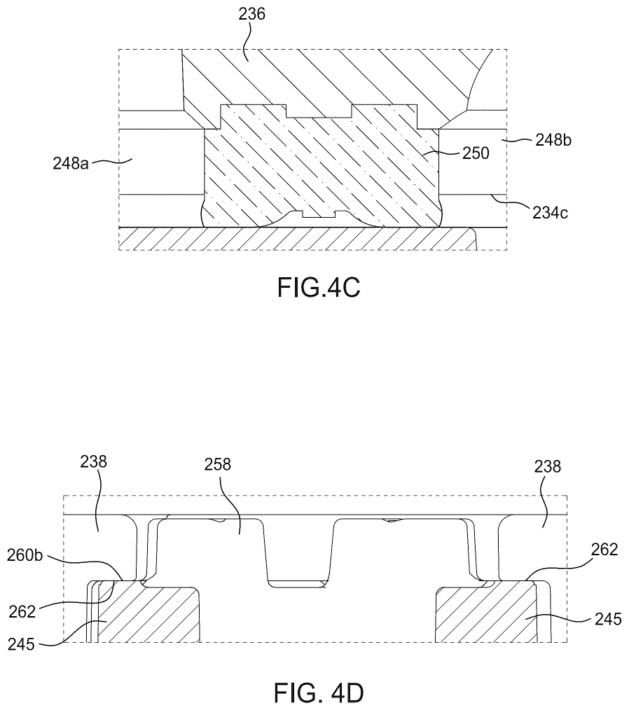

Valve body 236c includes a sealing material 250 at first end 240 which rotatably contacts backside surface 234c (shown in FIGS. 2G and 4A-4D) of base 206 for scaling first end 240 with backside surface 234c. Sealing between first end 240 and backside surface 234c forms a sealing interface between planar (e.g., non-cylindrical) surfaces. Because the sealing interface is on a longitudinal end (i.e., first end 240) of valve body 236c, this sealing arrangement may be referred to as end-sealing. In certain embodiments, sealing material 250 is formed from a rubber or elastomeric material (e.g., silicone rubber) which is bonded (e.g., overmolded) to valve body 236c at first end 240. In some other embodiments, valve body 236c and sealing material 250 may be integrally formed from the same material (e.g., high-density polyethylene).

Greater detail of the interaction between the sealing material 250 and the backside surface 234c may be had in FIGS. 4A-4C. Specifically, because the valve body 236 comprises a pair of passages 248a, 248b, the sealing material 250 when viewed in cross section forms a left and right portion at the outer circumference as detailed, for example, in FIG. 4B, as well as a center portion as detailed in FIG. 4C. As seen in FIG. 4B, the sealing material 250 which forms the left portion has a cross-sectional width of 1.52 mm, while the cross-sectional width of the center portion of the sealing material 250 seen in FIG. 4C is 3.56 mm with an indentation defined therein which reaches a maximum height of 0.15 mm. It is to be expressly understood that the right portion at the outer circumference is substantially identical to the left portion seen in FIG. 4B even though it is not explicitly described herein. In some embodiments, the scaling material 250 has an initial height of 1.52 mm, which in certain embodiments may be compressible when a force is applied.

A plurality of ports 252 are formed through partition 220 from backside surface 234 to frontside surface 222, as shown in FIG. 2G. In operation, valve body 236c is rotatable relative to backside surface 234c of base 206 to align each passage 248 with a corresponding port 252 (252a-c) of base 206 to open pressure and/or fluid communication between corresponding ones of the plurality of channels 216. A flow axis through each port 252 is parallel to longitudinal axis 246 of valve body 236c. A shape of each port 252 may correspond to a cross-section of each passage 248 of valve body 236c to help maintain flow therethrough. In certain embodiments, a cross-sectional shape of each passage 248 may be formed by continuing a shape of the corresponding port 252 as a swept surface through valve body 236c.

In the illustrated embodiments, five ports 252 are formed through partition 220, from the backside surface 234 to frontside surface 222, within each bore 230. However, there may be any suitable number of ports (e.g., two to seven ports) in each bore 230. In the illustrated embodiments, ports 252 include arc-shaped annular segments. In some other embodiments, ports 252 may be circular, round, oval, polygonal, square, any other suitable shape, or combinations thereof. In the illustrated embodiments, ports 252 have an equal flow area. In some other embodiments, ports 252 may have different flow areas. In the illustrated embodiments, ports 252 are uniformly spaced in the circumferential direction. In some other embodiments, ports 252 may have different spacing in the circumferential direction.

Retaining ring 238c has an annular body 254 with a center opening 256. Retaining ring 238c fits over and around valve body 236c such that a drive interface 258 (shown in FIG. 2G) of valve body 236c is received within center opening 256. In some embodiments, drive interface 258 engages a drive mechanism of console 100 for rotating valve body 236 about longitudinal axis 246. Annular body 254 includes multiple stepped portions having different outer dimensions. At least one portion of annular body 254 is disposed radially between outer cylindrical surface 244 of valve body 236c and cylindrical inner wall 232c of bore 230c. At least another portion of annular body 254 is disposed outside bore 230c. An outer shoulder 260a formed between the stepped portions of annular body 254 contacts backside 212 of base 206 when retaining ring 238c is partially or fully seated in bore 230c. As seen in FIG. 4D, an inner shoulder 260b formed between the stepped portions of annular body 254 contacts a backside surface 262 of valve body 236c radially surrounding drive interface 258 to apply a retention force on valve body 236c. In certain embodiments, a lubricant (e.g., silicone oil) may be used to facilitate relative rotation between interfacing surfaces of valve body 236c (e.g., backside surface 262) and retaining ring 238c (e.g., inner shoulder 260b), and/or between sealing material 250 and backside surface 234c. In certain embodiments, one or more of the interfacing surfaces may be impregnated with lubricant. In some embodiments, the lubricant may be any liquid that provides a lubricious and/or hydrophobic coating.

The retention force applied by the retaining ring 238 is applied in a direction parallel to longitudinal axis 246 (axially) and forces first end 240 of valve body 236c towards backside surface 234c of base 206 which compresses sealing material 250 against backside surface 234c thereby forming a seal between passages 248 and corresponding ports 252. In certain embodiments, sealing material 250 is compressed in the direction parallel to longitudinal axis 246 (axially) up to 34% of its total height, thereby compressing or reducing the overall height down to approximately 1 mm, when retaining ring 238c is fully seated in bore 230c. In certain embodiments, retaining ring 238c is coupled to base 206 using a solid-state welding technique (e.g., ultrasonic welding) so that no gap or space is present between the inner shoulder 260b of the retaining ring 238 and the backside surface 262 of the valve body 236. In some other embodiments, retaining ring 238 may for example be snap-fit, threaded, and/or adhered to base 206. In certain embodiments, the valve body 236c itself may be snap-fit, threaded, and/or adhered directly to base 206 without use of the retaining ring 238.

In certain embodiments, valve body 236 includes a hard stop feature that can be used to correlate a rotational state of valve body 236 with one of the base 206 or retaining ring 238 in order to ensure proper alignment between passages 248 and corresponding ports 252 during operation. In FIGS. 2F-2G, the hard stop feature includes a first profile 264 formed on cylindrical outer surface 244 of valve body 236 which is configured to contact a corresponding second profile 266 formed on annular body 254 of retaining ring 238. Further rotation of valve body 236 is prevented by contact between first profile 264 and corresponding second profile 266. As shown in FIG. 2G, second profile 266 is disposed in a corresponding notch 268 formed on cylindrical inner wall 232c of bore 230c so that rotational alignment of retaining ring 238c is fixed relative to base 206 which is necessary for functioning of the hard stop feature. In other embodiments, the second profile 266 may be formed directly on the base 206.

In certain other embodiments, the hard stop feature may comprise one or more optical indicators disposed on the valve body 236 and the retaining ring 238, respectively, for detection by optical sensors on the surgical console 100. For example, instead of physical contact being made between the valve body 236 and the retaining ring 238, the one or more optical indicators may indicate when a first maximum rotational position between the valve body 236 and retaining ring 238 is achieved, thereby indicating to the surgical console 100, or other system, the current alignment or rotational position the valve body 236 is in relative to the retaining ring 238.

To correlate a rotational state of valve body 236 with retaining ring 238 using the hard stop feature, valve body 236 may be rotated in a first direction (e.g., clockwise) about longitudinal axis 246 until first profile 264 contacts a first side of second profile 266 at which point a first rotational state is recorded. Then, valve body 236 may be rotated in a second opposite direction (e.g., counterclockwise) about longitudinal axis 246 until first profile 264 contacts a second opposite side of second profile 266 at which point a second rotational state is recorded. Because rotational alignment of retaining ring 238 is fixed relative to base 206 via the second profile being inserted into notch 268, alignment between passages 248 and corresponding ports 252 is precisely known at any rotational state between the first and second rotational states corresponding to the hard stop feature.

FIGS. 5A-5B are frontside elevation views of a portion of surgical cassette 200 of FIG. 2A illustrating two different valve positions, according to certain embodiments. In FIGS. 5A-5B, cover assembly 208 is omitted for clarity. An exemplary first valve rotational state of valve assembly 204c is illustrated in FIG. 5A. In the first valve rotational state, first passage 248a (indicated with dashed outline) of valve body 236c is aligned with first port 252a and fifth port 252e of venturi reservoir 282. When first passage 248a is aligned with both ports of venturi reservoir 282, fluid communication with venturi reservoir 282 is closed, and therefore, the first port 252a and fifth port 252e may be referred to as “closed” in the first valve rotational state. First port 252a is adjacent to fifth port 252e and is disposed through partition 220 of base 206 below level sensor area 289 like fifth port 252e as described above. In the first valve rotational state, second passage 248b (indicated with dashed outline) is aligned with a second port 252b corresponding to a second channel 216b and a third port 252c corresponding to a third channel 216c, thereby opening fluid communication between second channel 216b and third channel 216c. In the first valve rotational state, a sector of sealing material 250 in a clockwise direction relative to second passage 248b is aligned with a fourth port 252d, thereby closing fluid communication through fourth port 252d.

In certain embodiments, in the first valve rotational state, a hard prime may be performed on second channel 216b upstream of first pump assembly 202a (also referred to as an “aspiration path” of surgical cassette 200). In general, the hard prime involves building-up vacuum very high in the aspiration path and then suddenly opening the aspiration path to liquid from venturi reservoir 282. The sudden rush of liquid causes a high flow rate through the aspiration path and shears any trapped air bubbles from their positions. The trapped air bubbles are allowed to enter the main flow stream and are passed to a drain bag coupled to surgical cassette 200. In certain embodiments, the hard prime is an automated sequence performed by computer 103 of console 100. The hard prime may be performed as part of the surgical setup of surgical cassette 200. The hard prime provides lower and more repeatable total aspiration path compliance (improved vacuum responsiveness) of surgical cassette 200 and results in lower fluid volume surges during post-occlusion break surge events.

In certain embodiments, second channel 216b is in fluid communication with first pump assembly 202a (also referred to as an “aspiration pump”) and third channel 216c is in fluid communication with an aspiration line, e.g., of a surgical handpiece or other device for aspirating fluid from the eye (e.g., handpiece 112 shown in FIG. 1A). In such embodiments, the first rotational state corresponds to an aspiration state.

An example operation of valve assembly 204c is described below. In certain embodiments, occlusion or blockage of fluid flow through the handpiece (or other device) can occur during aspiration due to build-up of surgical material (e.g., lens fragments, debris, etc.) causing vacuum pressure to build-up between the handpiece and, e.g., first pump assembly 202a. With sufficient vacuum pressure build-up, breakage of the occlusion often occurs leading to a rapid surge of fluid being aspirated from the eye and into valve assembly 204c through third channel 216c, and in certain embodiments, including second channel 216b and first pump assembly 202a. This process is commonly referred to as post-occlusion break surge.

The occurrence of post-occlusion break surge may be mitigated using certain embodiments disclosed herein. In certain embodiments, post-occlusion break surge is indicated by a rapid pressure change, which may be detected using a pressure sensor in the handpiece or the surgical cassette 200. When post-occlusion break surge is detected, valve assembly 204c may be switched from the first valve rotational state of FIG. 5A to a second valve rotational state illustrated in FIG. 5B by rotating valve body 236c counterclockwise about longitudinal axis 246 by about 72 degrees (as indicated by the arrow). In the second valve rotational state, first passage 248a (indicated with dashed outline) of valve body 236c is aligned with fourth port 252d and fifth port 252c, thereby opening fluid communication between venturi reservoir 282 and third channel 216c. In the second valve rotational state, second passage 248b (indicated with dashed outline) is aligned with first port 252a and second port 252b, thereby opening fluid communication between venturi reservoir 282 and second channel 216b. In the second valve rotational state, the sector of sealing material 250 in a clockwise direction relative to second passage 248b is aligned with third port 252c, thereby closing fluid communication through third port 252c.

In embodiments with second channel 216b in fluid communication with first pump assembly 202a and third channel 216c in fluid communication with a surgical handpiece (as described above), other device, or surgical site, the second rotational state corresponds to a dual path venting state. Thus, fluid communication is open between first channel 216a of venturi reservoir 282 and first pump assembly 202a through second channel 216b. In the dual path venting state, fluid communication is also open between first channel 216a of venturi reservoir 282 and the surgical handpiece, other device, or surgical site through third channel 216c. Valve assembly 204c may be switched from the aspiration state shown in FIG. 5A to the dual path venting state shown in FIG. 5B by rotating valve body 236c counterclockwise about longitudinal axis 246 by about 72 degrees (as indicated by the arrow). Switching to the dual path venting state immediately when post-occlusion break surge is detected mitigates the resulting rapid aspiration of fluid from the eye by filling second channel 216b and third channel 216c with fluid before significant fluid flow from the eye occurs.

In the dual path venting state, first port 252a and fifth port 252e are opened simultaneously. Having first port 252a and fifth port 252e opened at the same time provides independent venting to venturi reservoir 282 of vacuum pressure built-up within corresponding channels 216b-c of base 206. For instance, vacuum pressure built-up in third channel 216c between fifth port 252e and the aspiration path is vented through fifth port 252e. Likewise, vacuum pressure built-up in second channel 216b between first port 252a and first pump assembly 202a is vented through first port 252a independently of the venting of the surgical site through channel 216c (e.g., the aspiration path). In certain embodiments, second channel 216b has a volume of about 4 cc (cubic centimeters), whereas the aspiration path (including third channel 216c) has a volume of only about 0.5 cc. Thus, the relatively high compliance of second channel 216b contributes a majority of the vacuum pressure volume that leads to the post-occlusion break surge. Therefore, the independent venting that is provided by switching fluid communication between second channel 216b leading to the pump and third channel 216c leading to the eye greatly reduces the post-occlusion break surge volume.

In certain embodiments, in response to detecting post-occlusion break surge, the surgical cassette 200 may be vented by pumping irrigation/infusion fluids through pump assembly 202b and downstream toward port 210c, while second valve assembly 204b and/or fourth valve assembly 204d are disposed in a rotational state corresponding to a venting state.

In certain embodiments, instead of the valve assembly 204c being rotated in the counterclockwise direction as shown in FIGS. 5A and 5B, the valve body 236c may instead be rotated clockwise about longitudinal axis 246, thereby venting second channel 216b and cutting off third channel 216c. In other words, rotating the valve body 236c clockwise would switch the fluid connection to the surgical site from the highly compliant path of the second channel 216b which is concurrently vented independently.

In certain embodiments, first port 252a and fifth port 252e of venturi reservoir 282 may be combined into one port. Similarly, third port 252c and fourth port 252d may be combined into one port. Thus, in some embodiments, there may be three ports utilized for dual path venting. In certain embodiments, a single path valve 402 may be used to vent channel 216c to venturi reservoir 216a. In other embodiments, channel 216b may be vented after channel 216c by continuing ration of the valve body 402.

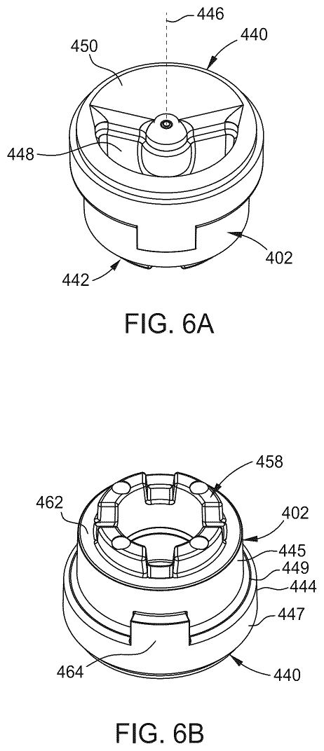

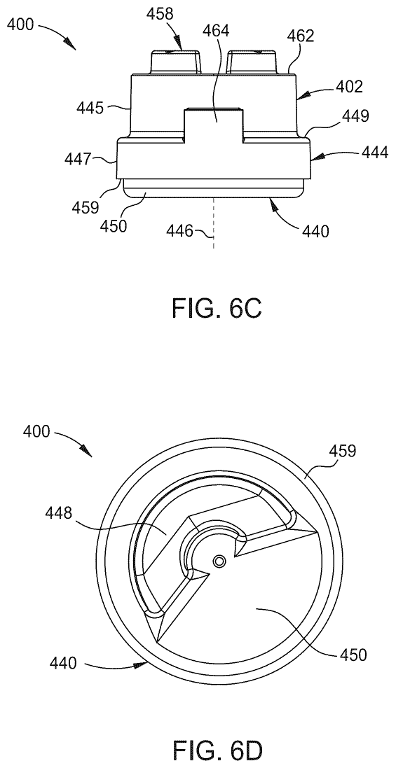

FIGS. 6A-6D illustrate an exemplary embodiment of a valve body 402 having only one passage 448. Valve body 402 has a first end 440, a second end 442, a cylindrical outer surface 444 connecting first end 440 and second end 442, and a longitudinal axis 446 orthogonal to first end 440. Only a single passage 448 is formed in valve body 402 at first end 440. Valve body 402 includes a sealing material 450 at first end 440. In FIG. 6A, passage 448 extends about longitudinal axis 446 in a circumferential direction by about 200-230°, or in certain embodiments by about 210-220°. In the illustrated embodiments, passage 448 is sized to simultaneously open fluid communication with three ports of base 206. In some other embodiments, passage 448 may be sized to simultaneously open fluid communication with any suitable number of ports (e.g., two, three, or four ports). In FIG. 6B, valve body 402 further comprises a drive interface 458 which engages a drive mechanism of console 100 for rotating valve body 402 about longitudinal axis 446. In certain embodiments, the second end 442 comprises a backside surface 462. Cylindrical outer surface 444 includes multiple stepped portions having different outer dimensions. In some embodiments, the cylindrical outer surface 444 is comprised of a base portion 445 separated from a collar portion 447 by a first shoulder 449, the collar portion 447 having a larger diameter relative to the base portion 445. In one embodiment, the base portion 445 has a diameter of approximately 13 mm and a longitudinal length of 5.21 mm, while the collar portion has a diameter of approximately 16 mm and a longitudinal length of 3.38 mm. Transitioning from the collar portion 447 to a scaling material 450 is a second shoulder 459.

In the illustrated embodiments, passage 448 includes an arc-shaped annular segment extending circumferentially about longitudinal axis 446. In certain embodiments, a cross-section of passage 448 may be circular, round, oval, polygonal, square, any other suitable shape, or combinations thereof. Terminal ends of the passage 448 are defined at first end 440 of valve body 402. In certain embodiments, a center axis of the passage 448 at the terminal ends is parallel to longitudinal axis 446. In certain embodiments, at least a portion of the passage 448, e.g., the portion between the terminal ends, is orthogonal to longitudinal axis 446. In certain embodiments, during fabrication, passage 448 are machined or molded in a direction parallel to longitudinal axis 446, e.g., starting from first end 440. In other words, an entire surface of the passage 448 is visible from first end 440 when viewed in a direction parallel to longitudinal axis 446. In the illustrated embodiments, the passage 448 includes a symmetrical flow area. In some other embodiments, the passage 448 may have an asymmetrical flow area to provide different flow characteristics across its arc length.

As mentioned above, valve body 402 includes the scaling material 450 at first end 440, which rotatably contacts backside surface 234c of base 206 for scaling first end 440 with backside surface 234c, similar to the valve body 236c shown in FIGS. 2G and 4A-4D. Sealing between first end 440 and backside surface 234c forms a sealing interface between planar (e.g., non-cylindrical) surfaces. Because the scaling interface is on a longitudinal end (i.e., first end 440) of valve body 402, this sealing arrangement may be referred to as end-scaling. In certain embodiments, scaling material 450 is formed from a rubber or elastomeric material (e.g., silicone) which is bonded (e.g., overmolded) to valve body 402 at first end 440. In some other embodiments, valve body 402 and scaling material 450 may be integrally formed from the same material (e.g., high-density polyethylene). Note that, in certain embodiments, scaling can occur between resolved surfaces of the surgical cassette 200 that make contact axially and/or radially with respect to one another.

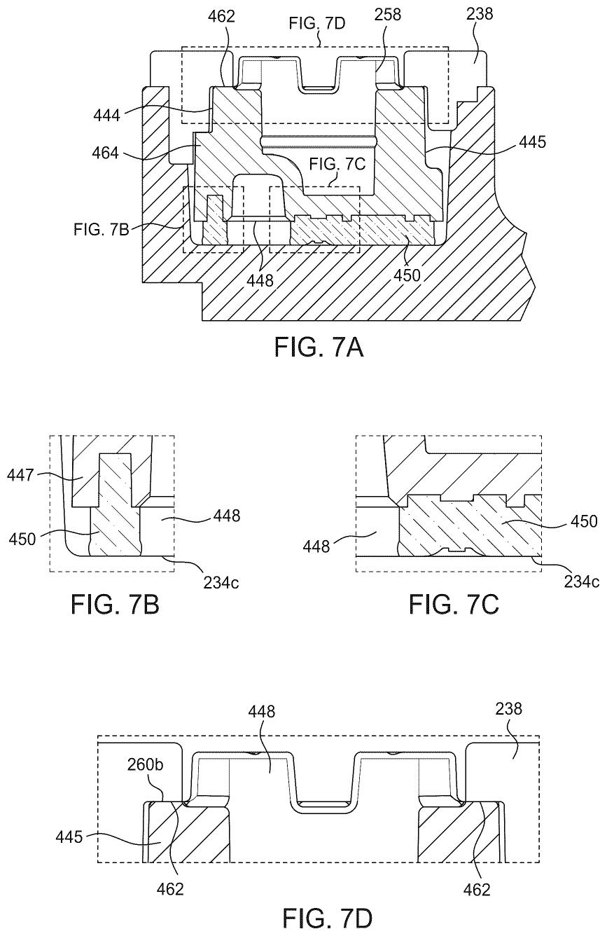

Greater detail of the interaction between the sealing material 450 of the single-passage valve body 402 and the backside surface 234c may be had in FIGS. 7A-7C. Specifically, because the valve body 402 comprises a single passage 448, the sealing material 450 when viewed in cross-section forms an off-center portion at the outer circumference as detailed, for example, in FIG. 7B, as well as a center portion detailed in FIG. 7C. As seen in FIG. 7B, the scaling material 450 which forms the off-center portion has a cross-sectional width of 1.52 mm, while the maximum cross-sectional width of the center portion of the sealing material 450 seen in FIG. 7C is 9.14 mm with an indentation defined therein which reaches a maximum height of 0.15 mm. In some embodiments, the scaling material 450 has an initial height of 1.52 mm, which in certain embodiments may be compressible when a force is applied.

In some embodiments, the retaining rings 238 disclosed and described above in relation to the valve bodies 236 may also be used in conjunction with the single-passage valve body 402. For example, at least one portion of annular body 254 of the retaining ring 238c is disposed radially between outer cylindrical surface 444 of valve body 402 and cylindrical inner wall 232c of bore 230c. At least another portion of annular body 254 is disposed outside bore 230c. An outer shoulder 260a formed between the stepped portions of annular body 254 contacts at least a portion of backside 212 of base 206 when, for example, retaining ring 238c is fully seated in bore 230c as seen in FIG. 7D. An inner shoulder 260b formed between the stepped portions of annular body 254 contacts a backside surface 462 of valve body 402 radially surrounding drive interface 258 to apply a retention force on valve body 402. In certain embodiments, a lubricant (e.g., silicone oil) may be used to facilitate relative rotation between interfacing surfaces of valve body 402 (e.g., backside surface 462) and retaining ring 238 (e.g., inner shoulder 260b), and/or between sealing material 250 and backside surface 234c. In certain embodiments, one or more of the interfacing surfaces may be impregnated with lubricant. In some embodiments, the lubricant may be any liquid that provides a lubricious and/or hydrophobic coating.

The retention force applied by the retaining ring 238c is applied in a direction parallel to longitudinal axis 446 (axially) and forces first end 440 of valve body 402 towards backside surface 234c of base 206 which compresses sealing material 450 against backside surface 234c thereby forming a seal between the passage 448 and corresponding ports 252. In certain embodiments, sealing material 450 is compressed in the direction parallel to longitudinal axis 446 (axially) up to 34% of its total height, thereby compressing or reducing the overall height down to approximately 1 mm, when retaining ring 238c is fully seated in bore 230c. In certain embodiments, retaining ring 238c is coupled to base 206 using a solid-state welding technique (e.g., ultrasonic welding) so that no gap or space is present between the inner shoulder 260b of the retaining ring 238c and the backside surface 462 of the valve body 402. In some other embodiments, retaining ring 238c may be snap-fit to base 206.

In certain embodiments, valve body 402 includes a hard stop feature that can be used to correlate a rotational state of valve body 402 with one of the base 206 or retaining ring 238c in order to ensure proper alignment between passages 248 and corresponding ports 252 during operation. The hard stop feature includes a first profile 464 formed on cylindrical outer surface 444 of valve body 402 which is configured to contact the corresponding second profile 266 formed on annular body 254 of retaining ring 238c. Further rotation of valve body 402 is prevented by contact between first profile 464 and corresponding second profile 266. Second profile 266 is disposed in a corresponding notch 268 formed on cylindrical inner wall 232c of bore 230c so that rotational alignment of retaining ring 238c is fixed relative to base 206 which is necessary for functioning of the hard stop feature. As best seen in FIG. 6C, in some embodiments the first profile 464 extends vertically upward from the collar portion 447 to a height of 1.8 mm or more, such as 2 mm or more, and has a width of approximately 4 mm or more, or 5 mm or more.

To correlate a rotational state of valve body 402 with retaining ring 238 using the hard stop feature, valve body 402 may be rotated in a first direction (e.g., clockwise) about longitudinal axis 446 until first profile 464 contacts a first side of second profile 266 at which point a first rotational state is recorded. Then, valve body 402 may be rotated in a second opposite direction (e.g., counterclockwise) about longitudinal axis 446 until first profile 464 contacts a second opposite side of second profile 266 at which point a second rotational state is recorded. Because rotational alignment of retaining ring 238c is fixed relative to base 206 via the second profile being inserted into notch 268, alignment between the passage 448 and corresponding ports 252 is precisely known at any rotational state between the first and second rotational states corresponding to the hard stop feature.

In some embodiments, the valve assembly's 204a-204d may comprise a combination of both multiple-passage valve bodies 236 and single-passage valve bodies 402. For example, first valve assembly 204a and third valve assembly 204c as seen in FIG. 2B may each comprise a multiple-passage valve body 236 which are each in pressure and/or fluid communication with first pump assembly 202a and port 210a to provide aspiration (suction) through port 210a and ventilation during an operation respectively, while the second valve assembly 204b and fourth valve assembly 204d each comprise a single-passage valve body 402 which are each in pressure and/or fluid communication with second pump assembly 202b and port 210c to provide irrigation (infusion) through port 210c and bypass during the same operation, respectively.

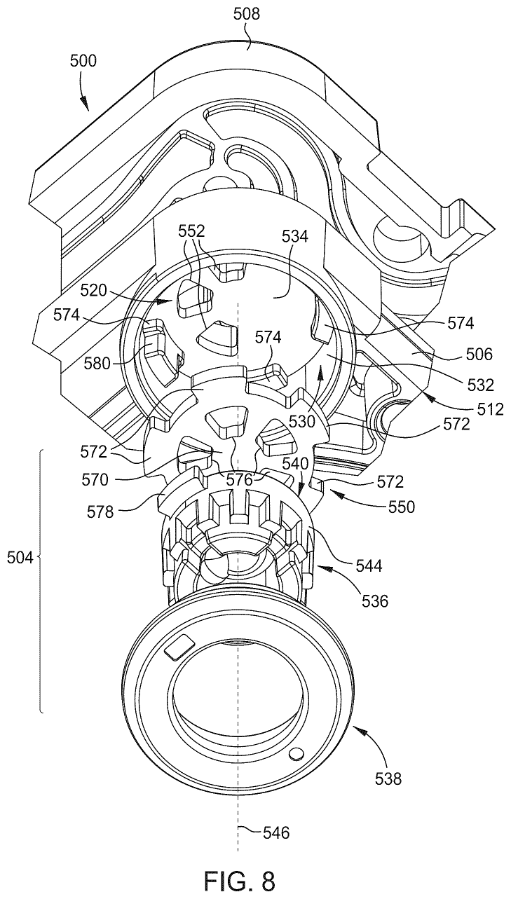

FIG. 8 is an exploded backside isometric view illustrating an alternative valve assembly, according to certain embodiments. Surgical cassette 500 and valve assembly 504 are similar to surgical cassette 200 and valve assembly 204c unless otherwise noted. Surgical cassette 500 generally includes a base 506, a cover assembly 508 coupled to base 506, and valve assembly 504 located on backside 512 of base 506 (which is visible in FIG. 8). Valve assembly 504 generally includes a valve body 536, a retaining ring 538, and a seal piece 550.

Seal piece 550 is disposed between first end 540 of valve body 536 and backside surface 534 of base 506. Seal piece 550 includes a backside surface 570 in sealing contact with first end 540 of valve body 534 and a frontside surface (which is not visible in FIG. 8) facing away from backside surface 570 in sealing contact with backside surface 534 of partition 520 of base 506. Valve body 536 is rotatable relative to seal piece 550 about longitudinal axis 546 of valve body 536. A plurality of tabs 572 on seal piece 550 interlock with a corresponding plurality of cutouts 574 within bore 530 of base 506 to rotatably fix seal piece 550 relative to base 506. The plurality of cutouts 574 are connected to backside surface 534 and cylindrical inner wall 532 of bore 530. Ports 576 (e.g., four shown) disposed through seal piece 550 correspond to ports 552 (e.g., three shown) in partition 520 of base 506.

Valve assembly 504 includes a hard stop feature between valve body 504 and base 506 in contrast to the hard stop feature of valve assembly 204c between valve body 236c and retaining ring 538c. In FIG. 8, valve body 504 has a first profile 578 formed on cylindrical outer surface 544 for contacting a corresponding second profile 580 formed on cylindrical inner wall 532 of base 506.

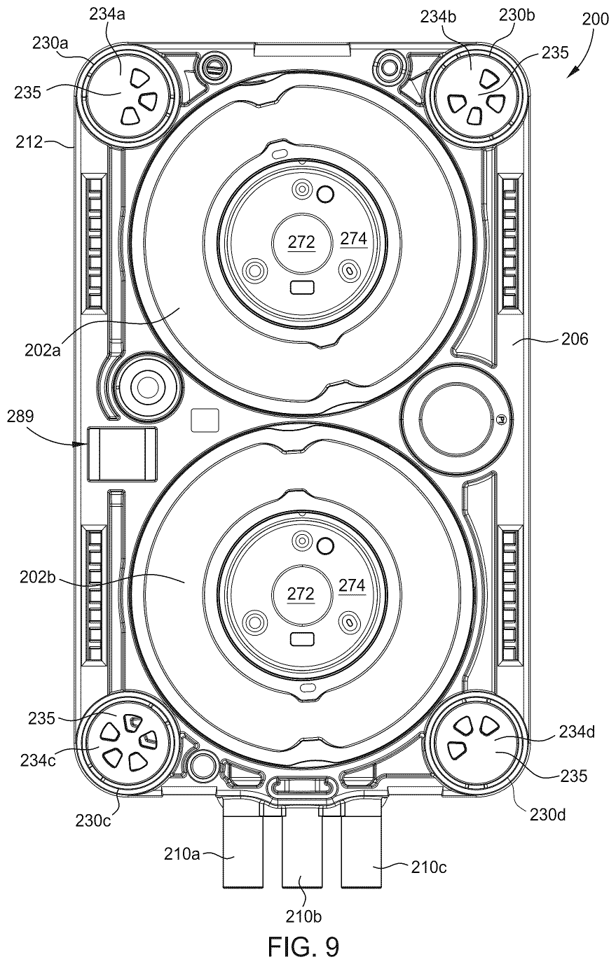

Turning to FIG. 9, the surgical cassette 200 is shown where each of the valve assemblies 204 have been removed from their corresponding bores 230a-230d to illustrate the respective backside surfaces 234a-234d disposed therein. In some embodiments, each of the backside surfaces 234a-234d comprises a surface finish or polish 235, which provides a texture or friction coefficient critical to minimizing the torque between the valve assemblies 204 and the base 206. In some embodiments, the surface finish is provided by an electrical discharge machining (EDM) process of the mold, however in other embodiments, other methods for etching, surface finishing, or polishing known in the art, including chemical etching and bead blasting, may be used.

The retention force applied by the retaining ring 238 which compresses the sealing material 250, 450 of the valve bodies 236, 402 against the backside surfaces 234a-234d ensures that a sufficient seal is formed between each of the valve assemblies 204 and the base 206 which is tight enough to prevent leaking of fluids but not so tight as to require excessive torque to rotate of the valve assemblies 204, while the lubricant disposed between the interacting surfaces of the retaining ring 238 and valve bodies 236, 402 and the surface finish provided on the backside surfaces 234a-234d in turn reduces the amount of torque required and increases the amount of abrasion resistance. In other words, the specific dimensions and materials used as disclosed above cooperate to provide a sufficient seal at each of its valve assemblies 204, which may be actuated using a minimum amount of torque. Using a minimum amount of torque not only lengthens the use life of the valve assemblies 204 but also provides significantly faster response for occlusion break surge mitigation as it eliminates the need for speed reducing gearboxes for any motors disposed in the fluidics subsystem 110 within the surgical console 100 that engage or interact with the surgical cassette 200.

In some embodiments, the surgical cassette 200 further comprises one or more features configured to interact with components of the surgical console 100 for facilitating identification of the type, serial number, batch number, etc., of surgical cassette 200 by the surgical console 100. In one embodiment, the features for facilitating identification of the surgical cassette 200 may also simultaneously facilitate measurements of fluid levels within the venturi reservoir 282 disposed within the surgical cassette 200 by the surgical console 100. In one embodiment, the features for facilitating identification of the surgical cassette 200 may also simultaneously facilitate the retrieving of different types of data relating to the surgical cassette 200 and/or the surgical console 100, such as, e.g., calibration data.

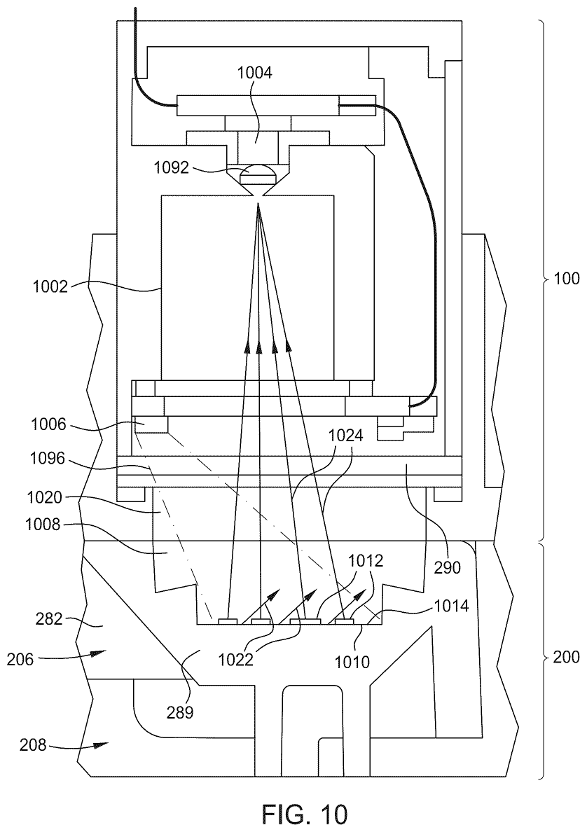

An example of the features for identifying the surgical cassette 200, as well as for retrieving pressure sensor calibration data from the surgical cassette 200, is illustrated in FIG. 10, which shows a magnified top down cross sectional view of the interface between the level sensor area 289 defined in the base 206 of the surgical cassette 200, and an image sensor area 1002 disposed in the fluidics subsystem 110 of the surgical console 100. The image sensor area 1002 comprises an image sensor 1004 which, in some embodiments, comprises a camera, or other image sensor/detector, and at least one visible light source 1006 that is configured or angled to emit at least one beam of visible light at an oblique angle relative to both a window 290 disposed within the surgical console 100 in front of the image sensor area 1002 and to the barcode surface 1010. The level sensor area 289 comprises a cavity 1008. The cavity 1008 comprises a barcode surface 1010 onto which a barcode 1016 is disposed. In one embodiment, the barcode 1016 is laser-etched onto the barcode surface 1010, namely with the barcode 1016 comprising a plurality of smooth surfaces 1014 intermingled or interspaced with a plurality of etched surfaces 1012 created by the laser. In one embodiment, the smooth surfaces 1014 of the barcode 1016 are comprised of smooth, dark, black, or other light absorbing plastic, however because the etched surfaces 1012 of the barcode 1016 have had their topmost layer roughened by a laser, each of the etched surfaces 1012 comprises a rough or uneven surface that can diffuse or scatter light.

In one embodiment, for the surgical console 100 to scan or read the barcode 1016 disposed within the surgical cassette 200, visible light 1020 is emitted from the at least one visible light source 1006 along an optical path 1096 that enters the cavity 1008 via the window 290 disposed within the surgical console 100 at an oblique angle relative to the window 290. After being transmitted through the window 290, the visible light 1020 strikes, or impinges upon, the barcode 1016 on the barcode surface 1010, the visible light 1020 illuminating both the smooth surfaces 1014 and the etched surfaces 1012 of the barcode 1016 at an oblique angle. The portions of the visible light 1020 which illuminate the smooth surfaces 1014 of the barcode 1016 are either reflected away from the image sensor 1004, as indicated by arrows 1022, or are absorbed by the material comprising the barcode surface 1010, while the portions of the visible light 1020 that illuminate the etched surfaces 1012 are diffused, some of which are directed back through the window 290 and through a lens 1092 that focuses the light for forming an image onto the image sensor 1004 as indicated by lines 1024.

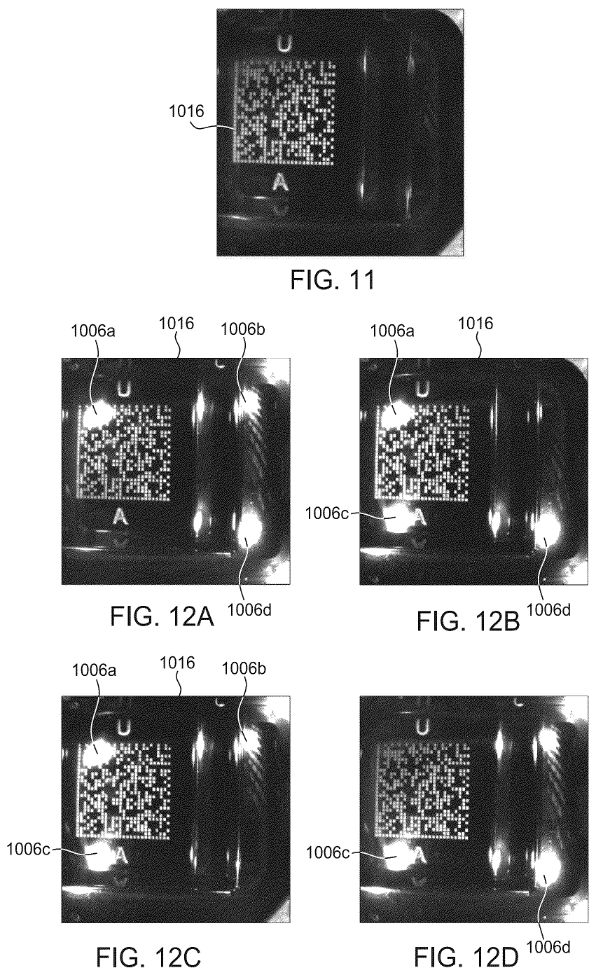

The end result, as seen in FIG. 11, is an image of the barcode 1016 comprised of light and dark portions, the light portions representing the light 1024 that was reflected back and focused onto the image sensor 1004 from the etched surfaces 1012, and the dark portions representing the light 1022 which was reflected away or absorbed by the smooth surfaces 1014. The surgical console 100, and particularly, computer 103 of the surgical console 100, can then “read” the image(s) of the barcode 1016 and thereby identify the type, and/or retrieve calibration data, of surgical cassette 200 currently inserted into the fluidics subsystem 110 of the surgical console 100 and ensure that the cassette 200 is the correct cassette for the procedure currently being performed on a patient, and retrieve the correct calibration data for irrigation and/or aspiration sensors in the surgical console 100 as coded into the barcode at the time of cassette fabrication.

In other words, the computer 103 receives information collected and relayed thereto from the image sensor 1004, e.g., image(s) of the barcode. The information is processed by the processor of computer 103 and mapped to one or more profiles of surgical cassette types/forms stored in the memory of computer 103 to identify the type of surgical cassette 200. Once the type of surgical cassette 200 is identified, the surgical console 100 may execute settings and/or task parameters associated with the type of surgical cassette 200 and/or associated calibration data. For example, the surgical console 100 may provide control of fluidics subsystem 110 according to the type of surgical cassette 200. In certain embodiments, the image sensor 1004 itself includes a processor that processes the barcode image(s) prior to relaying the data to the computer 103.

In one embodiment, the image sensor area 1002 comprises four different visible light sources each of which are configured to illuminate the barcode 1016 at an oblique angle as disclosed above. As seen in FIGS. 12A-12D, references 1006a-d correspond to the light reflected off barcode surface 1010 from each of the four different visible light sources, respectively. In a related embodiment, the four visible light sources are symmetrically disposed about the image sensor 1004, namely with at least two visible light sources disposed on each lateral side of the image sensor 1004, thereby disposing the image sensor 1004 in the middle or center of the disposed visible light sources. However when multiple visible light sources are used and an image is acquired by the image sensor 1004, the glare or amount of light that is reflected from each of the visible light sources 1006a-d can obfuscate the barcode 1016 and render it difficult for the user to detect. To solve this problem, the image sensor 1004 captures a sequence of images of the barcode 1016 with each image within the sequence having at least one of the visible light sources 1006a-d darkened or turned off while the remaining visible light sources 1006a-d are illuminated. For example, as seen in FIGS. 12A-12D four images are acquired by the image sensor 1004 with each one of the images comprising a different one of the plurality of visible light sources darkened or turned off, thereby producing a corresponding number of reflections 1006a-d. FIG. 12A is an illustration of a first image taken within the sequence in which a third visible light source is darkened, while a first visible light source, a second visible light source, and a fourth visible light source remain illuminated, thereby producing a corresponding first reflection 1006a, a second reflection 1006b, and a fourth reflection 1006d.

As seen in FIG. 12A, the light reflected from the illuminated visible light sources blocks or partially blocks the corresponding portions of the barcode 1016, while the portion of the barcode 1016 corresponding to where the third visible light source is disposed remains dark, thereby allowing the diffused light 1024 from the etched surfaces 1012 to clearly appear within the image. The process is then repeated for each of the remaining visible light sources, namely with FIG. 12B illustrating how the barcode 1016 appears when the second visible light source is darkened, FIG. 12C illustrating how the barcode 1016 appears when the fourth visible light source is darkened, and FIG. 12D illustrating how the barcode 1016 appears when the first visible light source is darkened.

After all images of the barcode 1016 are obtained, image processing software within the surgical console 100 combines all of the images seen in FIGS. 12A-12D to create a single image of the barcode 1016 as shown in FIG. 11 by selectively combining the darkened portions of each respective image of FIGS. 12A-12D into a single composite image as seen in FIG. 11 which comprises the most clearly received diffused light 1024 from each portion of the barcode 1016. While four visible light sources 1006a-d and four images are seen in FIGS. 12A-12D, it is to be expressly understood that fewer or additional images may be taken or that fewer or additional visible light sources may be used other than what is explicitly disclosed herein. For example, in one particular embodiment, the image sensor 1004 may only take two images, a first image where only the first and second visible light sources 1006a, 1006b are illuminated, and a second image where only the third and fourth visible light sources 1006c, 1006d are illuminated. The internal software within the surgical console 100 may then overlay or “stitch” the bottom portion of the first image to the top portion of the second image to create a single final image which comprises the clearest or most readable portions of the barcode 1016. In certain embodiments, a final image is provided consisting of three regions, namely a top region, a middle region, and a bottom region. In some embodiments, the bottom region will be from a first image, the top region will be from a second image, and the middle region will be a transition zone where the middle region comprises a blend of the first and second images. At a bottom edge of the middle region, this could, for example, be 100% from the first image and 0% from the second image which linearly transitions to 0% from the first image and 100% from the second image at a top edge of the middle region. In certain other embodiments, the final image of barcode 1016 is a combination of some, or all the images captured in a sequence by the image sensor 1004 such that the intensity of each pixel in the final composite image is the minimum, or second minimum, or third minimum of the intensities of the same pixel amongst the images in the sequence.

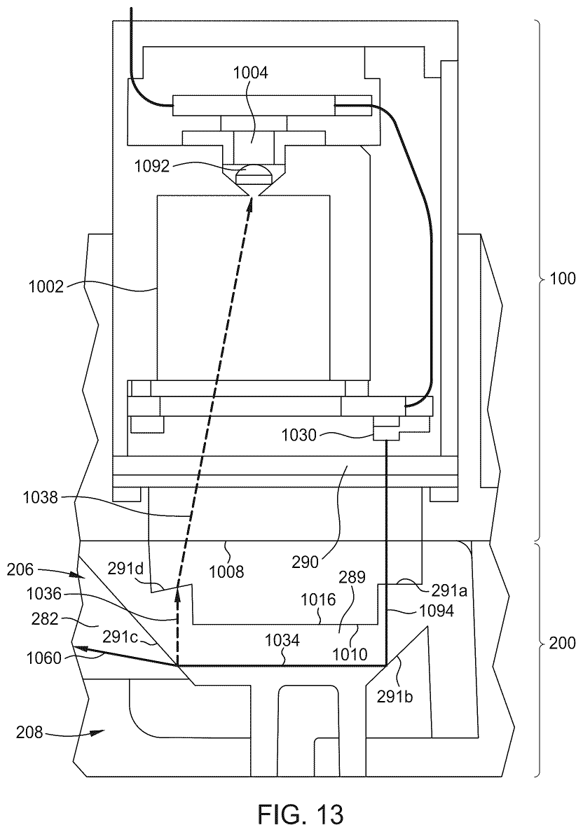

In some embodiments, features for determining a current fluid level within the venturi reservoir 282 are also disposed within the surgical cassette 200. As seen in FIG. 13, venturi reservoir 282 is adjacent the level sensor area 289 defined in base 206. For reference, vacuum port 283 shown in FIGS. 2C-2E is disposed above level sensor area 289. When the surgical cassette 200 is coupled to the surgical console 100, level sensor area 289 is disposed to provide an optical path 1094 from an infrared light source 1030 to the image sensor 1004 in surgical console 100 that is used to determine a fluid level in venturi reservoir 282. In certain embodiments, the image sensor 1004 is a single camera sensor or a complementary metal oxide semiconductor (CMOS) sensor.

Also shown in FIGS. 2C-2E is the cavity 1008, which is formed in backside 212 of base 206. The position of the cavity 1008 corresponds to the level sensor area 289. The relatively small size or footprint of level sensor area 289 enables the use of a more compact housing 205 of the surgical cassette 200 as compared to other designs. In certain embodiments, base 206 is comprised of plastic or plastic composite that is transparent to infrared light to allow infrared light impinging on surface 291a of base 206 to pass into, reflect internally twice at surfaces 291a-b (when fluid is absent), and back out (at surface 291d) towards the image sensor 1004 for infrared sensing of the fluid level in the venturi reservoir 282 (described further below). In certain embodiments, the venturi reservoir 282 is a volume of space formed between the base 206 and a cover 208a. In certain embodiments, portions of cover 208a and manifold 208b overlaying level sensor area 289 are infrared and/or visible light blocking to prevent signal interference caused by light passing through cover 208a and manifold 208b. In certain embodiments, portions of cover 208a and manifold 208b overlaying level sensor area 289 are transparent to visible light, thereby allowing a user viewing the frontside of housing 205 to observe the fluid level in level sensor area 289.

As shown in FIG. 2D, a lens feature 297 is disposed on a backside surface of manifold 208b. Lens feature 297 diverts ambient light (including infrared and visible wavelengths) away from the path leading to the image sensor 1004 to improve sensing of the fluid level in level sensor area 289. In some other embodiments, lens feature 297 includes an array of alternating angled surfaces that overlay at least part of level sensor area 289.

Other aspects may also be implemented to help block ambient light from reaching level sensor area 289. In certain embodiments, filter 287 is positioned and/or shaped to overlay at least a portion of level sensor area 289. In other embodiments, a drain bag is applied to a frontside of manifold 208b in a position that overlays at least a portion of level sensor area 289. A double-sided adhesive that blocks ambient light may be used to adhere the drain bag to manifold 208b. An example adhesive area 298 is illustrated in FIG. 2C inside the dashed line on the frontside of manifold 208b. In some other embodiments, one or more opaque stickers are applied to cover 208a and/or manifold 208b in positions that overlay at least a portion of level sensor area 289. In other embodiments, ink printing onto the cover 208a and/or manifold 208b may be utilized.

Returning now to FIG. 13, the cavity 1008 of the level sensor area 289 comprises a plurality of surfaces 291a-d, which in some embodiments includes a normal surface 291a and angled surfaces 291b, 291c, and 291d. In certain embodiments, the plurality of surfaces 291a-d may comprise any combination of flat, angled, or curved configurations while maintaining the same functionality. For example, in one embodiment, the surface 291a is flat and normal to the optical path 1094 of at least one incoming infrared light beam emitted by the infrared light source 1030 disposed within the image sensor area 1002 of the surgical console 100. After being transmitted through the surface 291a, the infrared (IR) light contacts the first angled surface 291b which is aligned with both the infrared light source 1030 in console 100 and, in some embodiments, the venturi reservoir 282. The first angled surface 291b comprises a polish or texture, as well as an angular orientation (if flat) or curvature, to help direct the IR light from the IR light source 1030 towards the second angled surface 291c. In other words, infrared light that is emitted from console 100 reflects off the first angled surface 291b, passes from the viewer's right to left along the optical path 1094 as indicated by arrow 1034, then partially reflects off the second angled surface 291c (which is flat or curved) away from the viewer back towards console 100 along the optical path 1094 as indicated by the dashed line arrow 1036. The portion of infrared light that is reflected back towards console 100 passes through the third angled surface 291d which is orientated to redirect the reflected IR light through the window 290 and lens 1092 disposed within the surgical console 100 and onto the image sensor 1004 in surgical console 100 (for reference, see the optical path 1094 as indicated by dashed line arrow 1038). In some embodiments, each angled surface 291b, 291c is oriented at about 45 degrees in relation to a plane of base 206 so that the infrared light reflected back towards console 100 is reversed, or rotated by about 180 degrees, in relation to the infrared light emitted from console 100. However, in some other embodiments, angled surfaces 291b, 291c and angled surface 291d are oriented at other angles that correspond to the positions and/or orientations of the infrared light source 1030, lens 1092, and image sensor 1004. In some other embodiments, surfaces 291a and 291d are essentially the same and continuous with backside 212 of base 206. In other embodiments, surface 1016 is also continuous with backside 212 of base 206.

Detection of the relative fluid level within the venturi reservoir 282 works based on the difference in index of refraction of air compared to liquid at surface 291c. In one embodiment, as IR light strikes the second angled surface 291c, a majority of the first portion of the IR light that is incident where there is liquid present on surface 291c, is refracted at the solid-liquid interface and continues along path 1060 into the venturi reservoir 282, while a second or remaining portion of the IR light, incident where there is no liquid present on surface 291c, undergoes total internal reflection off of the second angled surface 291c and is then directed to the angled surface 291d. Following this process, an image of surface 291c is projected by the lens 1092 into the image sensor 1004. Regions of the surface that are in contact with liquid appear dark; and regions that are in contact with air appear light. This image can be analyzed to determine the fluid level. The proportion or amount of IR light which enters the fluid, 1060, to the proportion or amount of IR light which undergoes total internal reflection, 1036, and is reflected off of the second angled surface 291c depends upon the amount of fluid currently contained within the venturi reservoir 282. Specifically, the portion of incoming IR light 1034 which strikes a corresponding surface region of the venturi reservoir 282 which has fluid disposed behind it will refract into the venturi reservoir 282 and thus not ever be detected by the image sensor 1004. Meanwhile, the portion of incoming IR light 1034 which strikes a corresponding surface portion of the venturi reservoir 282 which does not have fluid behind it will instead undergo total internal reflection and reflect off of the second angled surface 291c and then be subsequently detected by the image sensor 1004. The net effect therefore is that IR light is absorbed where there is fluid in the venturi reservoir 282 and reflected where it is not, thereby providing a means to determine the exact current amount of fluid within the venturi reservoir 282. In certain other embodiments, both surfaces 291b and 291c may be adjacent to the venturi reservoir so that both provide conditional reflectivity depending on fluid being absent. In other embodiments, only surface 291b is adjacent to the venturi reservoir.

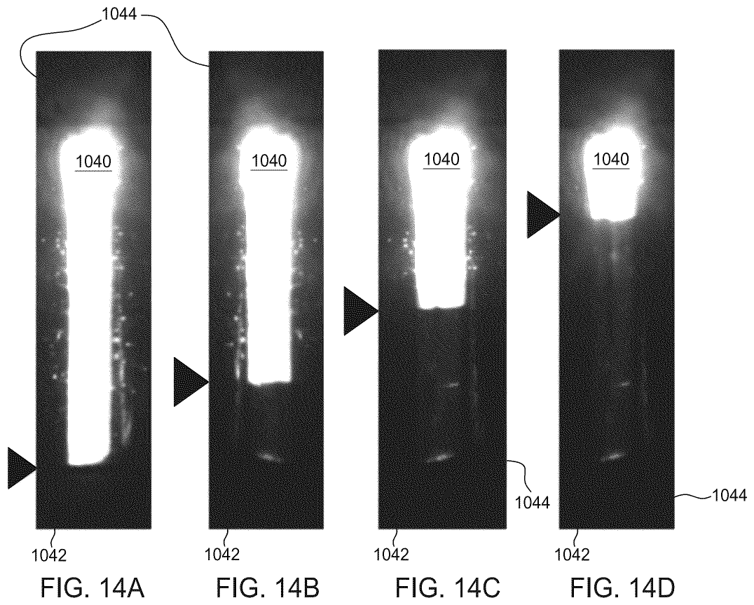

FIGS. 14A-D illustrate a series of example outputs of the image sensor 1004 when detecting multiple different fluid levels within the venturi reservoir 282. As seen in FIG. 14A, a majority of IR light has been detected by the image sensor 1004, thereby providing a reading with a light portion 1040 which is the majority of a fluid level meter 1044 (corresponding to the amount of light reflected from second angled surface 291c) and indicating that the venturi reservoir 282 is empty or nearly empty. In FIG. 14B, a large portion of IR light has been detected by the image sensor 1004, thereby providing a reading with a light portion 1040 which occupies a top ¾ of the reading and a dark portion 1042 which occupies the remaining bottom ¼ of the fluid level meter 1044 and indicating that the venturi reservoir 282 is approximately ¼ full of fluid. In FIG. 14C, a portion of IR light has been detected by the image sensor 1004, thereby providing the fluid level meter 1044 with a light portion 1040 which occupies a top ½ of the fluid level meter 1044 and a dark portion 1042 which occupies the remaining bottom ½ of the fluid level meter 1044 and indicating that the venturi reservoir 282 is approximately ½ full of fluid. In FIG. 14D, only a small portion of IR light has been detected by the image sensor 1004, thereby providing the fluid level meter 1044 with a light portion 1040 which occupies a top ¼ of the fluid level meter 1044 and a dark portion 1042 which occupies the remaining bottom ¾ of the fluid level meter 1044 and indicating that the venturi reservoir 282 is approximately ¾ full of fluid.

Accordingly, improved ophthalmic surgical cassettes, valve assemblies therefor, means for being read by an image sensor disposed within the surgical console, and methods of use thereof are provided herein.

### EXAMPLE EMBODIMENTS

Embodiment 1: A surgical cassette configured to be coupled to a surgical console for surgical irrigation, infusion, aspiration, or suction during a surgical procedure, the surgical cassette comprising: a housing, comprising: a base; a cover coupled to the base, wherein the cover and the base each at least partially define one or more channels disposed between the cover and the base; a plurality of ports formed in the base, each of the plurality of ports in fluid communication with at least one of the one or more channels; a plurality of bores formed in the base, each of the plurality of bores configured to receive one of a plurality of valve assemblies; and at least one pump landing formed in the base, the at least one pump landing configured to receive at least one pump assembly; the plurality of valve assemblies disposed within the plurality of bores, the plurality of valve assemblies configured to control fluid flow in the one or more channels of the housing; and the at least one pump assembly disposed within the at least one pump landing, the at least one pump assembly configured to provide a source of pressure or vacuum to the one or more channels of the housing.
Embodiment 2: The surgical cassette of Embodiment 1, wherein: the plurality of bores comprises at least two bores; the plurality of valve assemblies comprises two valve assemblies disposed within the two bores; and each of the two bores and the two valve assemblies is disposed at an edge of the base.
Embodiment 3: The surgical cassette of Embodiment 2, wherein: the at least one pump landing comprises a plurality of pump landings; the at least one pump assembly comprises a plurality of pump assemblies disposed within the plurality of pump landings; the plurality of pump landings comprises two adjacent pump landings; each of the two adjacent pump landings is substantially disposed at a center of the base' and the plurality of pump assemblies comprises two pump assemblies disposed within the two pump landings
Embodiment 4: The surgical cassette of Embodiment 1, wherein: the at least one pump landing comprises a plurality of pump landings; the at least one pump assembly comprises a plurality of pump assemblies disposed within the plurality of pump landings; the plurality of pump landings comprises two adjacent pump landings; each of the two adjacent pump landings is substantially disposed at a center of the base; and the plurality of pump assemblies comprises two pump assemblies disposed within the two pump landings.
Embodiment 5: The surgical cassette of Embodiment 1, wherein: each of the two bores and the two valve assemblies is disposed at a corner of the base; the at least one pump landing comprises a plurality of pump landings; the at least one pump assembly comprises a plurality of pump assemblies disposed within the plurality of pump landings; the plurality of pump landings comprises two adjacent pump landings; each of the two adjacent pump landings is substantially disposed at a center of the base; and the plurality of pump assemblies comprises two pump assemblies disposed within the two pump landings.
Embodiment 6: The surgical cassette of Embodiment 1, wherein: the plurality of bores comprises at least four bores, and wherein the plurality of valve assemblies comprises four valve assemblies disposed within the four bores; the at least one pump landing comprises a plurality of pump landings; the at least one pump assembly comprises a plurality of pump assemblies disposed within the plurality of pump landings; the plurality of pump landings comprises two adjacent pump landings, wherein each of the two adjacent pump landings is substantially disposed at a center of the base; the plurality of pump assemblies comprises two pump assemblies disposed within the two pump landings.
Embodiment 7: The surgical cassette of Embodiment 1, wherein: each of the four bores and the four valve assemblies is disposed at a corner of the base; the at least one pump landing comprises a plurality of pump landings; the at least one pump assembly comprises a plurality of pump assemblies disposed within the plurality of pump landings; the plurality of pump landings comprises two adjacent pump landings; each of the two adjacent pump landings is substantially disposed at a center of the base; and the plurality of pump assemblies comprises two pump assemblies disposed within the two pump landings.
Embodiment 8: A surgical cassette configured to be coupled to a surgical console for surgical irrigation or aspiration during a surgical procedure, the surgical cassette comprising: a housing, comprising: a partition separating a first surface of the housing from a second surface of the housing; a plurality of ports formed in the first surface of the housing; and a plurality of channels adjoining the second surface of the housing and in fluid communication with the plurality of ports, wherein one or more of the channels is in fluid communication with a source of pressure or vacuum; and one or more valve assemblies coupled to the housing and configured to control fluid communication between the plurality of channels of the housing, wherein each of the one or more valve assemblies comprises: a valve body having a first end, a second end, and a cylindrical surface connecting the first end and second end, wherein: one or more passages are formed in the first end of the valve body, the first end of the valve body seals with the first surface of the housing, the valve body is rotatable about a first axis orthogonal to the first end and relative to the first surface of the housing to align the one or more passages with one or more of the plurality of ports of the housing to open fluid communication between corresponding ones of the plurality of channels, and a drive interface is formed on the second end of the valve body and configured to engage a drive mechanism for rotating the valve body.
Embodiment 9: The surgical cassette of Embodiment 8, wherein the valve body comprises a sealing material at the first end for sealing the first end with the first surface of the housing, wherein the sealing material rotatably contacts the first surface of the housing.
Embodiment 10: The surgical cassette of Embodiment 9, wherein each of the one or more valve assemblies further comprises a retaining ring coupled to the housing, wherein the retaining ring applies a retention force on the valve body in a direction parallel to the first axis forcing the first end of the valve body towards the first surface of the housing and compressing the sealing material therebetween.
Embodiment 11: The surgical cassette of Embodiment 8, wherein the housing further comprises a cover, and wherein the plurality of channels are sealingly enclosed through contact between a second surface of the housing and a corresponding face extending from an inner surface of the cover.
Embodiment 12: The surgical cassette of Embodiment 8, wherein a first valve assembly of the one or more valve assemblies comprises a first valve body having two passages formed in the first end.
Embodiment 13: The surgical cassette of Embodiment 12, wherein when the first valve body is in a first rotational state: a first passage of the two passages is aligned with a first port corresponding to a first channel of the housing and a fifth port corresponding to the first channel to close fluid communication with the first channel, and a second passage of the two passages is aligned with a second port corresponding to a second channel and a third port corresponding to a third channel to open fluid communication between the second channel and third channel.
Embodiment 14: The surgical cassette of Embodiment 13, wherein the first channel is in fluid communication with a fluid reservoir, the second channel is in fluid communication with a first pump assembly disposed in the housing and configured to provide a source of vacuum or pressure, and the third channel is in fluid communication with an aspiration device for aspirating fluid from an eye, and wherein the first rotational state corresponds to an aspiration state.
Embodiment 15: The surgical cassette of Embodiment 14, wherein: when a post-occlusion break surge is detected, the first valve body is rotated about the first axis to a second rotational state comprising a dual path venting state for mitigating a rapid aspiration of fluid from the eye, when the first valve body is in the second rotational state, the first passage is aligned with the fifth port and a fourth port corresponding to the third channel of the housing to open fluid communication between the fluid reservoir and a surgical handpiece, and when the first valve body is in the second rotational state, the second passage is aligned with the first port and second port to open fluid communication between the fluid reservoir and the first pump assembly.
Embodiment 16: The surgical cassette of Embodiment 12, wherein the first valve assembly is coupled to an aspiration pump, further comprising: a second valve assembly coupled to the aspiration pump; a third valve assembly coupled to an irrigation pump; and a fourth valve assembly coupled to the irrigation pump.
Embodiment 17: The surgical cassette of Embodiment 8, wherein the valve body further comprises a first profile formed on the cylindrical surface for contacting a corresponding second profile formed on at least one of the housing or a retaining ring coupled to the housing, wherein further rotation of the valve body is prevented by contact between the first profile and the corresponding second profile.
Embodiment 18: The surgical cassette of Embodiment 10, wherein each retaining ring is coupled to the housing through a solid-state weld.
Embodiment 19: The surgical cassette of Embodiment 8, wherein the drive mechanism comprises a direct drive mechanism.
Embodiment 20: The surgical cassette of Embodiment 8, wherein each of the one or more valve assemblies further comprises a seal piece disposed between the first end of the valve body and the first surface of the housing, wherein the seal piece is rotatably fixed relative to the housing.
Embodiment 21: The surgical cassette of Embodiment 8, wherein an entire surface of each of the one or more passages is visible from the first end of the valve body when viewed in a direction parallel to the first axis.
Embodiment 22: The surgical cassette of Embodiment 8, wherein a cross-section of each of the one or more passages of the valve body corresponds to a shape of the plurality of ports of the housing to help maintain laminar flow therethrough.
Embodiment 23: A surgical cassette coupled to a surgical console for surgical irrigation or aspiration during a surgical procedure, the surgical cassette comprising: a housing, comprising: a base; a cover coupled to the base; and a venturi reservoir disposed inside the housing between the base and cover, the venturi reservoir comprising: a level sensor area defined in the base for determining a fluid level in the venturi reservoir; a first port disposed through the base on a first side of the level sensor area; a second port disposed through the base on a second side of the level sensor area; and a plurality of air baffles disposed within the venturi reservoir, the plurality of air baffles configured to divert at least some air bubbles entering the venturi reservoir from each of the first and second ports in the base away from the level sensor area.
Embodiment 24: The surgical cassette of Embodiment 23, wherein the plurality of air baffles is integral with the base and contact the cover when the cover is coupled to the base.
Embodiment 25: The surgical cassette of Embodiment 23, wherein the surgical cassette is coupled to an external vacuum source disposed in the surgical console, the external vacuum source configured to apply vacuum pressure to the venturi reservoir.
Embodiment 26: The surgical cassette of Embodiment 25, wherein the cover comprises a vacuum port in pressure communication between the external vacuum source and the venturi reservoir, the vacuum port disposed on the second side of the level sensor area.
Embodiment 27: The surgical cassette of Embodiment 26, further comprising: a manifold coupled to the cover; and a filter disposed between the cover and the manifold, the filter in pressure communication between the external vacuum source and the vacuum port in the cover for blocking fluid from entering the external vacuum source.
Embodiment 28: The surgical cassette of Embodiment 27, wherein the cover and the base respectively comprise openings in a direction perpendicular to a plane of the base, the openings in pressure communication between the external vacuum source and the filter.
Embodiment 29: The surgical cassette of Embodiment 27, wherein the housing further comprises a catch reservoir defined between the cover and the manifold to catch fluid that leaks through the filter.
Embodiment 30: The surgical cassette of Embodiment 26, wherein the venturi reservoir further comprises a liquid baffle disposed within the venturi reservoir on the second side of the level sensor area and configured to reduce or prevent liquid from entering the vacuum port in the cover, wherein the liquid baffle is integral with the base and contacts the cover when the cover is coupled to the base.
Embodiment 31: The surgical cassette of Embodiment 27, further comprising a filter disposed between the cover and the manifold, wherein the cover comprises a filter drain hole in fluid communication between the filter and the venturi reservoir.
Embodiment 32: The surgical cassette of Embodiment 31, wherein the filter is hydrophobic.
Embodiment 33: The surgical cassette of Embodiment 23, wherein the first port in the base is in fluid communication with an aspiration line of a surgical handpiece.
Embodiment 34: The surgical cassette of Embodiment 33, wherein the venturi reservoir further comprises a third port disposed through the base on the first side of the level sensor area, the third port in fluid communication with an aspiration pump disposed in the housing.
Embodiment 35: The surgical cassette of Embodiment 34, wherein the first and third ports are configured to close to build-up vacuum upstream of the aspiration pump to help remove air bubbles trapped in an aspiration path of the surgical cassette.
Embodiment 36: The surgical cassette of Embodiment 34, wherein the first and third ports are configured to be opened simultaneously to vent vacuum pressure to the venturi reservoir, wherein: vacuum pressure built-up between the first port and the aspiration line is vented through the first port, and vacuum pressure built-up between the third port and the aspiration pump is vented through the third port independently of venting of the aspiration line.
Embodiment 37: The surgical cassette of Embodiment 23, wherein the base comprises an infrared transparent material corresponding to the level sensor area.
Embodiment 38: A surgical console configured to couple with a surgical cassette, the surgical console comprising: at least one light source, wherein: when the surgical cassette is coupled to the surgical console, the at least one light source is configured to emit at least one beam of light that: impinges upon at least one of a plurality of surfaces disposed on the surgical cassette, and reflects onto an image sensor; the image sensor configured to detect the at least one beam of light reflected from the at least one of the plurality of surfaces; a memory comprising executable instructions; and a processor in data communication with the memory and configured to execute the instructions to: detect a type of the surgical cassette based on sensor data received from the image sensor, and retrieve pressure sensor calibration parameters based on the sensor data received from the image sensor.
Embodiment 39: The surgical console of Embodiment 38, wherein the at least one light source comprises a visible light source.
Embodiment 40: The surgical console of Embodiment 38, wherein the plurality of surfaces disposed on the surgical cassette comprises: a barcode surface; and a barcode disposed on the barcode surface, wherein the barcode comprises at least one smooth surface and at least one etched surface.
Embodiment 41: The surgical console of Embodiment 40, wherein the light source is configured to emit the at least one beam of light at an oblique angle relative to the barcode surface.
Embodiment 42: The surgical console of Embodiment 41, wherein the image sensor is configured to detect the at least one beam of light reflected off of the etched surface of the barcode.
Embodiment 43: The surgical console of Embodiment 38, wherein the at least one light source is an infrared (IR) light source.
Embodiment 44: The surgical console of Embodiment 38, wherein: the plurality of surfaces comprise: a first surface configured to allow the at least one beam of light to enter the surgical cassette, and a second surface configured to reflect and redirect the at least one beam of light approximately 90 degrees towards a third surface, the third surface is configured to reflect and redirect the light approximately 90 degrees towards a fourth surface, the fourth surface is configured to allow the light to exit the surgical cassette towards an image sensor, the third surface forms at least a portion of a reservoir disposed within the surgical cassette, the image sensor is configured to detect at least a portion of light reflected off of the third surface, the portion of detected light corresponding to a volume of empty space within the reservoir, and the processor is further configured to determine a fluid level within the reservoir based on sensor data received from the image sensor, the sensor data indicative of the volume of empty space within the reservoir.
Embodiment 45: A method for determining a type of a surgical cassette when the surgical cassette is coupled to a surgical console, the method comprising: emitting at least one beam of light from a light source disposed in the surgical console, wherein: the at least one beam of light illuminates a bar code defined in a surface disposed on the surgical cassette; at least a portion of the at least one beam of light reflects off of at least a portion of the surface; detecting the reflected portion of the at least one beam of light with an image sensor disposed in the surgical console; and determining a type of the surgical cassette based on sensor data generated by the image sensor as a result of the detecting.
Embodiment 46: The method of Embodiment 45, wherein: emitting light from the light source disposed in the surgical console comprises emitting light from the light source at an oblique angle relative to the surface disposed on the surgical cassette, the at least one beam of light illuminating the bar code comprises the at least one beam of light illuminating at least one smooth surface of the bar code and at least one etched surface of the bar code, and the portion of the surface corresponds to the at least one etched surface of the bar code.
Embodiment 47: A method for determining a fluid level within a surgical cassette when the surgical cassette is coupled to a surgical console, the method comprising: emitting at least one beam of light from a light source disposed in the surgical console, wherein: the at least one beam of light is emitted into a partition defined within the surgical cassette; the at least one beam of light is directed along an optical path in the partition to a surface of the partition adjacent to a reservoir disposed within the surgical cassette; at least a portion of the at least one beam of light reflects off of the surface of the partition adjacent to the reservoir; detecting the reflected portion of the at least one beam of light with an image sensor disposed in the surgical console; and determining the fluid level within the reservoir based on sensor data generated by the image sensor as a result of the detecting, the sensor data indicative of a volume of empty space within the reservoir.
Embodiment 48: The method of Embodiment 47, wherein: the at least one beam of light is infrared (IR) light; the light source is an IR light source disposed in the surgical console; and the partition is an IR transparent window disposed on the surgical cassette.
Embodiment 49: The method of Embodiment 47, further comprising: passing the reflected portion of the at least one beam of light through an angled surface disposed within the surgical cassette configured to redirect the reflected portion of the at least one beam of light to the image sensor.
Embodiment 50: The surgical console of Embodiment 44, wherein: the fourth surface is adjacent to the barcode surface as viewed from the image sensor.
Embodiment 51: The surgical console of Embodiment 44, wherein: the surgical cassette is comprised of a base and a cover, the plurality of surfaces form a portion of the base, and the base and cover adjoin to form the reservoir.
Embodiment 52: The surgical console of Embodiment 51, wherein: the base material is visibly opaque while transparent to at least a portion of the infrared spectrum.
Embodiment 53: A surgical cassette for coupling to a surgical console for surgical irrigation or aspiration during a surgical procedure, the surgical cassette comprising: a housing; at least one valve assembly rotatably coupled to the housing and configured to control fluid communication between a plurality of channels within the housing; and at least one retaining ring coupled to the housing, where the at least one valve assembly comprises: a valve body having a first end, a second end, and a cylindrical surface connecting the first end and second end; and one or more passages defined in the first end of the valve body, wherein the valve body is configured to engage with the housing and the at least one retaining ring to form a seal with a first surface of the housing.
Embodiment 54: The surgical cassette of Embodiment 53, wherein the valve body of the at least one valve assembly comprises an elastomeric sealing material at the first end for sealing the first end with the first surface of the housing, wherein the elastomeric sealing material rotatably contacts the first surface of the housing.
Embodiment 55: The surgical cassette of Embodiment 53, wherein a first valve assembly of the at least one valve assembly comprises a first valve body having two passages formed in the first end.
Embodiment 56: The surgical cassette of Embodiment 53, wherein the housing comprises a first surface, the first surface comprising at least one backside surface corresponding to the at least one valve assembly, wherein the at least one backside surface comprises a surface finish configured to provide a predetermined amount friction between the first end of the valve body and the first surface of the housing.
Embodiment 57: The surgical cassette of Embodiment 55, wherein when the first valve body is in a first rotational state: a first passage of the two passages is aligned with a first port corresponding to a first channel of the housing and a fifth port corresponding to the first channel to close fluid communication with the first channel, and a second passage of the two passages is aligned with a second port corresponding to a second channel and a third port corresponding to a third channel to open fluid communication between the second channel and third channel.
Embodiment 58: The surgical cassette of Embodiment 54, wherein the at least one retaining ring is configured to engage with the at least one valve assembly to compress the elastomeric sealing material against the first surface of the housing.
Embodiment 59: The surgical cassette of Embodiment 54, wherein the cylindrical surface of the valve body comprises: a base portion; a collar portion joined to the base portion by a first shoulder; and a stop disposed on the collar portion, wherein the collar portion is joined to the elastomeric sealing material by a second shoulder.
Embodiment 60: The surgical cassette of Embodiment 53, wherein the at least one retaining ring comprises an inner shoulder that is configured to engage with a backside surface of the at least one valve assembly.
Embodiment 61: The surgical cassette of Embodiment 58, wherein the at least one retaining ring is configured to compress the elastomeric sealing material against the first surface of the housing up to 19% of a total height of the elastomeric sealing material.
Embodiment 62: The surgical cassette of Embodiment 54, wherein the elastomeric sealing material is overmolded to the valve body of the at least one valve assembly.
Embodiment 63: The surgical cassette of Embodiment 59, wherein the stop disposed on the collar portion comprises a height of at least 2 mm (millimeters) and a width of at least 4 mm.
Embodiment 64: The surgical cassette of Embodiment 56, wherein the surface finish configured to provide a predetermined amount friction between the first end of the valve body and the first surface of the housing is provided by an electrical discharge machining process.
Embodiment 65: The surgical cassette of Embodiment 55, wherein a second valve assembly of the at least one valve assembly comprises a second valve body having one passage formed in the first end.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What is claimed is:

1. A surgical cassette configured to be coupled to a surgical console for surgical irrigation, infusion, aspiration, or suction during a surgical procedure, the surgical cassette comprising:
a housing, comprising:
a base;
a cover coupled to the base, wherein the cover and the base each at least partially define one or more channels disposed between the cover and the base;
a plurality of ports formed in the base, each of the plurality of ports in fluid communication with at least one of the one or more channels;
a plurality of bores formed in the base, each of the plurality of bores configured to receive one of a plurality of valve assemblies; and
at least one pump landing formed in the base, the at least one pump landing configured to receive at least one pump assembly;
the plurality of valve assemblies disposed within the plurality of bores, the plurality of valve assemblies configured to control fluid flow in the one or more channels of the housing; and
the at least one pump assembly disposed within the at least one pump landing, the at least one pump assembly configured to provide a source of pressure or vacuum to the one or more channels of the housing.

2. The surgical cassette of claim 1, wherein the at least one pump landing comprises a plurality of pump landings, and wherein the at least one pump assembly comprises a plurality of pump assemblies disposed within the plurality of pump landings.

3. The surgical cassette of claim 2, wherein the plurality of pump landings comprises two pump landings adjacent to each other, and wherein the plurality of pump assemblies comprises two pump assemblies disposed within the two pump landings.

4. The surgical cassette of claim 2, wherein the plurality of pump landings and the plurality of pump assemblies are centrally disposed on the base.

5. The surgical cassette of claim 1, wherein the plurality of bores comprises at least two bores, and wherein the plurality of valve assemblies comprises two valve assemblies disposed within the two bores.

6. The surgical cassette of claim 5, wherein each of the two bores and the two valve assemblies is disposed at a corner of the base.

7. The surgical cassette of claim 5, wherein each of the two bores and the two valve assemblies is disposed at a center of the base.

8. The surgical cassette of claim 1, wherein the plurality of bores comprises at least four bores, and wherein the plurality of valve assemblies comprises four valve assemblies disposed within the four bores.

9. The surgical cassette of claim 8, wherein each of the four bores and the four valve assemblies is disposed at a corner of the base.

10. The surgical cassette of claim 8,
wherein the at least one pump landing comprises a plurality of pump landings, and wherein the at least one pump assembly comprises a plurality of pump assemblies disposed within the plurality of pump landings; and
wherein the plurality of bores and the plurality of valve assemblies surround the plurality of pump assemblies.

11. The surgical cassette of claim 1, wherein each of the plurality of valve assemblies comprises a rotary valve configured to route fluid flow selectively between the one or more channels of the housing.

12. The surgical cassette of claim 1, wherein the plurality of ports comprises an aspiration/suction port and an irrigation/infusion port.

13. The surgical cassette of claim 12, wherein at least one of the plurality of valve assemblies is in fluid communication with the aspiration/suction port, and wherein at least another one of the plurality of valve assemblies is in fluid communication with the irrigation/infusion port.

14. The surgical cassette of claim 12, wherein the at least one pump assembly is in fluid communication with the aspiration/suction port.

15. The surgical cassette of claim 12, wherein the at least one pump assembly is in fluid communication with the irrigation/infusion port.
